(19) United States
(12) Patent Application Publication
(10) Pub. No.: US 2023/0165679 A1
(43) Pub. Date: Jun. 1, 2023

(54) VALVE DELIVERY SYSTEM

(71) Applicant: SHIFAMED HOLDINGS, LLC, Campbell, CA (US)

(72) Inventors: Ryan William BOYD, Santa Cruz, CA (US); Andrew BACKUS, Santa Cruz, CA (US); Keke LEPULU, Menlo Park, CA (US); Noah GOLDSMITH, Campbell, CA (US)

(21) Appl. No.: 17/995,776

(22) PCT Filed: Apr. 8, 2021
(86) PCT No.: PCT/US2021/026463

§ 371 Date: Oct. 7, 2022

Related U.S. Application Data
(60) Provisional application No. 63/007,243, filed on Apr. 8, 2020.
(60) Provisional application No. 63/039,898, filed on Jun. 16, 2020.

(51) Int. Cl. A61F 2/24; A61F 2/95

(52) U.S. Cl. CPC A61F 2/2427; A61F 2/9517

(57) ABSTRACT

A method for treating a diseased native valve in a patient includes tracking a positioning tool in a first configuration and a valve delivery device over a tether into a heart where the tether is connected to a spiral anchor in the heart; activating the positioning tool from the first configuration to a second configuration that is stiffer than the first configuration; adjusting, with the positioning tool, a position of the spiral anchor relative to a valve positioned within the delivery device; and deploying a valve from the delivery device to an expanded position within the spiral anchor.

### CROSS REFERENCE TO RELATED APPLICATIONS

This application claims priority to U.S. Provisional Patent Application No. 63/007,243, filed Apr. 8, 2020, and to U.S. Provisional Application No. 63/039,898, filed Jun. 16, 2020, the entireties of which are incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves are passive one-way valves that open and close in response to differential pressures. Patients with valvular disease have abnormal anatomy and/or function of at least one valve. For example, a valve may suffer from insufficiency, also referred to as regurgitation, when the valve does not fully close and allows blood to flow retrograde. Valve stenosis can cause a valve to fail to open properly. Other diseases may also lead to dysfunction of the valves. While medications may be used to treat the disease, in many cases the defective valve may need to be repaired or replaced at some point during the patient's lifetime. Existing valves and surgical repair and/or replacement procedures may have increased risks, limited lifespans, and/or are highly invasive. Some less-invasive transcatheter options are available, though these options are generally limited to aortic valve procedures, are limited in their patient-to-patient flexibility, and often take longer than desired to implant. It would therefore be desirable to provide a less invasive procedure for repair and replacement of heart valves, including the mitral valve, quicker surgical methods, and/or prosthetic valves that can accommodate a variety of individual patients.

Additionally, existing valve repair/replacement procedures are often complicated and time-consuming. Currently available procedures often require the placement of more than one component—for example, a prosthetic valve and a mechanism to anchor it to the native anatomy. Such procedures generally utilize multiple delivery catheters to carry the various components and delivery of each component separately to the valve, which can be time-consuming (particularly if components are delivered sequential), complicated, and/or dangerous. For example, some devices provide rotational anchoring elements to capture the native anatomy such as the chordae tendineae in order to reduce delivery time. However, such anchoring elements, often by design, capture and pull the chordae along during their rotation, which can torque or otherwise stress and damage the chordae during deployment of the anchor elements, resulting in the need for additional medical interventions for the patient. Moreover, such anchoring elements may require extrusion from a low-profile (e.g., elongated) delivery configuration to an expanded configuration at or near the native valve. In at least some instances, extrusion of the anchoring elements can be complicated and may not reliably deploy into the correct expanded configuration relative to the delivery device. Incorrect deployment may result in additional time to retract and re-deploy the anchoring element, more complicated anchoring procedures, and/or damage to the native tissue. It would therefore be desirable to provide quicker, less-complicated, less dangerous, and more reliably deployable valve assemblies for valvular replacement and repair.

### SUMMARY

In general, in one embodiment, a method for treating a diseased native valve in a patient includes: tracking a positioning tool in a first configuration and a valve delivery device over a tether into a heart where the tether is connected to a spiral anchor in the heart; activating the positioning tool from the first configuration to a second configuration that is stiffer than the first configuration; adjusting, with the positioning tool, a position of the spiral anchor relative to a valve positioned within the delivery device; and deploying a valve from the delivery device to an expanded position within the spiral anchor.

This and other embodiments can include one or more of the following features. The step of adjusting, with the positioning tool, a position of the spiral anchor relative to a valve positioned within the delivery device can include pulling the positioning tool proximally. The method can further include advancing the positioning tool until a distal end of the positioning tool engages with a distal engagement element on the tether. The method can further include rotating the positioning tool until the distal end is clocked with respect to the distal engagement element. The step of activating the positioning tool from the first configuration to the second configuration can include bending the positioning tool. Bending the distal end of the positioning tool can include tensioning the tether so as to compress the distal end of the positioning tool. The method can further include fixing a position of the positioning tool relative to a handle of the valve delivery device prior to bending the distal end of the positioning tool. The method can further include pulling proximally on the handle with the positioning tool fixed thereto to adjust a position of both the anchor and the valve relative to the heart. The step of adjusting, with the positioning tool, the position of the spiral anchor can include moving the spiral anchor axially towards a native mitral valve annulus. The step of tracking a valve delivery device over a tether into a heart can include tracking the valve delivery device over a tether into a first chamber of the heart. The method can further include advancing a distal end of the valve delivery device through a native valve annulus to a second chamber of the heart prior to deploying the valve. The step of tracking a valve delivery device over a tether into a heart can include tracking a monorail lumen of the valve delivery device over the tether.

In general, in one embodiment, a delivery system for delivering and positioning a valve prosthesis within a diseased native valve includes a valve delivery catheter, a tether channel extending within or alongside the valve delivery catheter, and a positioning tool configured to extend through the tether channel and over the tether. The tether channel is configured to receive a tether that is attached to a spiral anchor. The positioning tool includes a first configuration and a second configuration. A stiffness of the positioning tool in the second configuration is greater than a stiffness of the positioning tool in the first configuration.

This and other embodiments can include one or more of the following features. The positioning tool in the second configuration can include a fixed bend. The positioning tool can include a plurality of cutouts in a distal portion thereof. The cutouts can enable flexing of the positioning tool in the first configuration. The cutouts can be fixed relative to one another in the second configuration. A distal engagement element of the tether can be configured to compress the positioning tool to place or maintain the positioning tool in the second configuration. The distal engagement element can be beveled. The delivery system can further include a proximal control having an actuator configured to control tension on the tether so as to compress the positioning tool. The actuator can include a knob configured to enable sliding of a tether clamp relative to the positioning tool. The delivery system can further include a proximal control having a rotation mechanism configured to rotationally orient the positioning tool with respect to the tether. The tether channel can extend along an exterior of the valve delivery catheter in a monorail configuration. The delivery system can further include a proximal control having an actuator configured to control a relative position of the spiral anchor and the valve by moving the positioning tool. The actuator can include a knob configured to enable sliding of the proximal control relative to a handle of the valve delivery catheter.

In general, in one embodiment, a method for treating a diseased native valve in a patient includes: tracking a valve delivery device over a tether into a heart where the tether is connected to a spiral anchor in the heart; adjusting, with the tether, a position of the spiral anchor relative to a valve positioned within the valve delivery device; and deploying a valve from the valve delivery device to an expanded position within the spiral anchor.

This and other embodiments can include one or more of the following features. The step of adjusting, with the tether, a position of the spiral anchor relative to a valve positioned with the valve delivery device can include pulling proximally with the tether. The step of tracking a valve delivery device over a tether can further include tracking a positioning tool over the tether. The step of adjusting, with the tether, a position of the spiral anchor relative to a valve can further include adjusting with the positioning tool. The method can further include advancing the positioning tool over the tether until a distal end of the positioning tool engages with a distal engagement element on the tether. The method can further include rotating the positioning tool until the distal end is clocked with respect to the distal engagement element. The method can further include bending a distal end of the positioning tool prior to the step of adjusting a position of the spiral anchor. Bending the distal end of the positioning tool can include tensioning the tether so as to compress the distal end of the positioning tool. The method can further include fixing a position of the positioning tool relative to a handle of the valve delivery device prior to bending the distal end of the positioning tool. The method can further include pulling proximally on the handle with the positioning tool fixed thereto to adjust a position of both the anchor and the valve relative to the heart. The step of adjusting, with the tether, the position of the spiral anchor relative to a valve positioned within the valve delivery device can include moving the spiral anchor axially towards a native mitral valve annulus. The step of tracking a valve delivery device over a tether into a heart can include tracking the valve delivery device over a tether into a first chamber of the heart. The method can further include advancing a distal end of the valve delivery device through a native valve annulus to a second chamber of the heart prior to deploying the valve. The step of tracking a valve delivery device over a tether into a heart can include tracking a monorail lumen of the valve delivery device over the tether.

In general, in one embodiment, a delivery system for delivering and positioning a valve prosthesis within a diseased native valve includes a valve delivery catheter, a tether channel extending within or alongside the valve delivery catheter, and a proximal control having an actuator configured to control a relative position of the spiral anchor and the valve by moving the tether. The tether channel is configured to receive a tether that is attached to a spiral anchor.

This and other embodiments can include one or more of the following features. The actuator can include a knob configured to enable sliding of the proximal control relative to a handle of the valve delivery catheter. The delivery system can further include a positioning tool configured to extend within the tether channel over the tether. The actuator can be further configured to move the positioning tool. The proximal control can further include a second actuator configured to adjust a position of the tether with respect to the positioning tool. The second actuator can include a knob configured to enable sliding of a tether clamp relative to the positioning tool. The second actuator can be configured to pull proximally on the tether to compress the positioning tool. A distal portion of the positioning tool can include a plurality of cutouts. The cutouts can enable flexing of the positioning tool when the positioning tool is in a first configuration. The cutouts can be fixed relative to one another when the positioning tool is in a second configuration. A distal end of the positioning tool can be configured to engage with a distal engagement element on the tether. The distal engagement element can be beveled. The tether channel can extend along an exterior of the valve delivery catheter in a monorail configuration.

In general, in one embodiment, a method for treating a diseased native valve in a patient includes: tracking a valve delivery device and a positioning tool over a tether where the tether is connected to a spiral anchor in the heart; bending the positioning tool so as to position a distal end of the tether within a plane of the spiral anchor; adjusting, with the positioning tool, a position of the spiral anchor relative to a valve positioned within the delivery device; and deploying a valve from the delivery device to an expanded position within the spiral anchor.

This and other embodiments can include one or more of the following features. The step of adjusting, with the positioning tool, a position of the spiral anchor relative to a valve positioned within the delivery device can include pulling the positioning tool proximally. The method can further include advancing the positioning tool until a distal end of the positioning tool engages with a distal engagement element on the tether. The method can further include rotating the positioning tool until the distal end is clocked with respect to the distal engagement element. The step of bending the distal end of the positioning tool can include tensioning the tether so as to compress the distal end of the positioning tool. The method can further include fixing a position of the positioning tool relative to a handle of the valve delivery device prior to bending the distal end of the positioning tool. The method can further include pulling proximally on the handle with the positioning tool fixed thereto to adjust a position of both the anchor and the valve relative to the heart. Adjusting the position of the spiral anchor can include moving the spiral anchor axially towards a native mitral valve annulus. The step of tracking a valve delivery device over a tether into a heart can include tracking the valve delivery device over a tether into a first chamber of the heart. The method can further include advancing a distal end of the valve delivery device through a native valve annulus to a second chamber of the heart prior to deploying the valve. The step of tracking a valve delivery device over a tether into a heart can include tracking a monorail lumen of the valve delivery device over the tether. Bending can include bending approximately 90 degrees.

In general, in one embodiment, a delivery system for delivering and positioning a valve prosthesis within a diseased native valve includes a valve delivery catheter, a tether channel extending within or alongside the valve delivery catheter, and a positioning tool configured to extend through the tether channel and over the tether. The tether channel is configured to receive a tether that is attached to a spiral anchor. The positioning tool is configured to bend to a fixed configuration so as to position a distal end of the tether within a plane of the spiral anchor.

This and other embodiments can include one or more of the following features. The bend can be approximately 90 degrees. A distal portion of the positioning tool can include a plurality of cutouts. The cutouts can enable flexing of the positioning tool when the positioning tool is in a flexible configuration. The cutouts can be fixed relative to one another when the positioning tool is in the fixed configuration. A distal engagement element of the tether can be configured to compress the positioning tool when the positioning tool is bent to the fixed position. The distal engagement element can be beveled. The delivery system can further include a proximal control having an actuator configured to control tension on the tether so as to compress the positioning tool. The actuator can include a knob configured to enable sliding of a tether clamp relative to the positioning tool. The delivery system can further include a proximal control having a rotation mechanism configured to rotationally orient the positioning tool with respect to the tether. The tether channel can extend along an exterior of the valve delivery catheter in a monorail configuration. The delivery system can further include a proximal control having an actuator configured to control a relative position of the spiral anchor and the valve by moving the positioning tool. The actuator can include a knob configured to enable sliding of the proximal control relative to a handle of the valve delivery catheter.

In general, in one embodiment, a delivery system for delivering a prosthesis to a diseased valve includes an outer sheath having a central axis, a valve retention member at a distal end of the outer sheath and configured to hold a valve, and a tether lumen extending parallel with the outer sheath and configured to receive a tether. A distal end of the tether lumen is positioned proximal to a distal end of the outer sheath.

This and other embodiments can include one or more of the following features. The delivery system can further include a tether configured to extend through the tether lumen and configured to attach to an anchor of the valve prosthesis. The delivery system can further include a positioning tool configured to be carried on the tether. A distal portion of the tether can be configured to interact with a distal end of the positioning tool. A proximal end of the distal portion of the tether can be configured to interact with the distal end of the positioning tool. A proximal end of the distal portion of the tether can include a bevel configured to interact with a corresponding bevel on the distal end of the positioning tool. A distal portion of the positioning tool can include a plurality of cutout portions along a side of the distal portion. A distal portion of the positioning tool can be configured to bend when compressed. The bend can include a 90° bend. The valve retention member can be positioned within the outer sheath and can include one or more tabs configured to mate with corresponding cutouts in the retainer. The tether and positioning tool can be configured to correctly orient an anchor of the prosthesis relative to a mitral annulus and chordae of a patient. The tether lumen can be positioned within the outer sheath. The tether lumen can be positioned along on an outer wall of the outer sheath in a monorail configuration. The tether lumen can be positioned off-axis relative to the outer sheath. The delivery system can further include a tether capture mechanism distal to the tether lumen. The tether capture mechanism can be configured to releasably hold a distal portion of a tether. The tether capture mechanism can be configured to releasably hold the distal portion on-axis relative to the outer sheath. The delivery system can further include a nosecone distal to the outer sheath. The nosecone can include a distal tip that is off-axis relative to a central axis of the outer sheath. The valve retention member can include a valve capsule distal to the outer sheath.

In general, in one embodiment, a method for treating a diseased native valve in a patient includes tracking a delivery device over a tether into a first chamber of a heart, advancing the distal end of the delivery device through the native valve annulus, and releasing a valve retention member of the delivery device to deploy the valve in the native valve annulus. The tether is connected to an anchor near a native valve annulus in the heart. The tether extends from a distal end of a port that is positioned proximal to the distal end of the delivery device.

This and other embodiments can include one or more of the following features. The method can further include inserting a proximal end of the tether into the port. The method can further include tracking the tether proximally through a handle of the delivery device. The method can further include tracking the tether proximally through a handle of the delivery device until it fixes to a latching mechanism in the handle. The method can further include advancing a positioning tool over the tether until a distal end of the positioning tool interacts with a distal portion of the tether. The method can further include compressing a distal portion of the positioning tool. The method can further include bending a distal portion of the positioning tool to apply tension to the anchor. The method can further include positioning the anchor using the tether and positioning tool. Tracking the delivery device over a tether into a first chamber of a heart can include tracking a monorail lumen along the exterior of the delivery device over the tether. The method can further include, prior to tracking the delivery device over the tether into the first chamber of the heart, moving a portion of the tether from a position off-axis relative to a sheath of the delivery device to a position on-axis relative to the delivery sheath and holding the portion of the tether on-axis with a tether capture mechanism. The method can further include releasing the tether from the tether capture mechanism after tracking the delivery device over the tether into the first chamber of the heart.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1F show a method of delivering an anchor for a valve prosthesis to a native valve annulus.

FIGS. 2A-2J show a method of delivering a valve prosthesis to an anchor previously placed near a native valve annulus.

FIGS. 3A-3C show various views of the interaction between a distal end of a tether and a distal end of a positioning tool.

FIGS. 4A-4C show a tether and positioning tool being used to position an anchor relative to a valve delivery catheter.

FIG. 5 shows a valve delivery catheter system with a positioning tool control.

FIGS. 6A-6L show a method of use of the valve delivery catheter system of FIG. 5.

FIG. 7 shows an exemplary embodiment of a tether latching mechanism.

FIGS. 8A and 8B show a method of releasing a tether from within the tether latching mechanism of FIG. 7.

FIGS. 9A-9D show a method of catching a tether within the tether latching mechanism of FIG. 7.

FIG. 10 shows the interaction between tabs on a valve prosthesis and a valve retainer of a valve delivery catheter.

FIGS. 11A-11G show a method of delivering a valve prosthesis to an anchor previously placed near a native valve annulus.

FIG. 12 shows a valve delivery catheter for delivering a valve prosthesis.

FIG. 13 shows a handle for a valve delivery catheter.

FIGS. 14A-14B show examples of a failed and a successful septal crossing, respectively.

FIGS. 15A-15C show a valve delivery catheter including a catch mechanism.

FIGS. 16A-16D show a method of catching and releasing an anchor using the mechanism shown in FIGS. 15A-15C.

FIGS. 17A-17C show another embodiment of a valve delivery catheter including a catch mechanism.

FIGS. 18A-18F show a method of catching and releasing an anchor using the sheath and mechanism of FIGS. 17A-17C.

FIGS. 19A-19C show various views of a positioning tool control.

FIGS. 20A-20C show various views of a drive train of the positioning tool control of FIGS. 19A-19C.

FIGS. 21A-21L show a method of using the positioning tool control shown in FIGS. 19A-20C.

FIGS. 22A-22B show another embodiment of a positioning tool control.

FIGS. 23A-23I show a method of using the positioning tool control of FIGS. 22A-22B.

### DETAILED DESCRIPTION

Described herein are devices and methods for use in delivering a valve, for example during a mitral valve replacement. The devices and methods can be used in conjunction with a previously placed anchor that is configured to fix the valve in position within the anatomy. In some embodiments, the devices and methods can be configured to position the anchor with respect to the anchor and fix or lock the position of the anchor with respect to the valve.

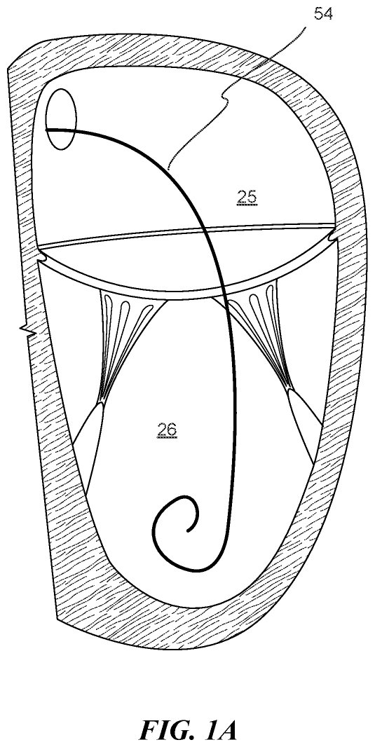

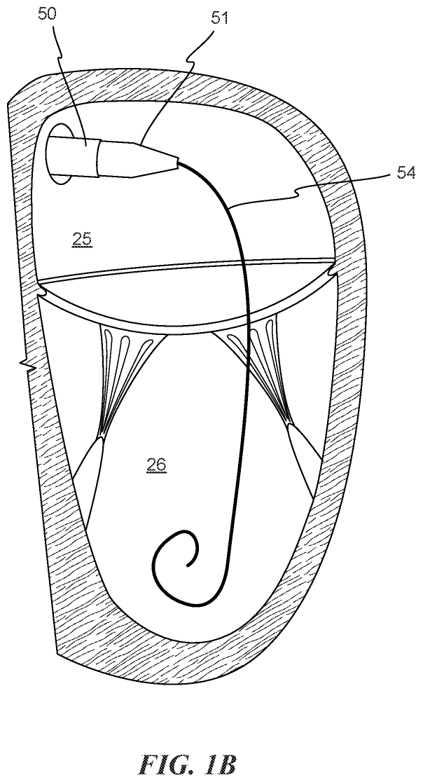

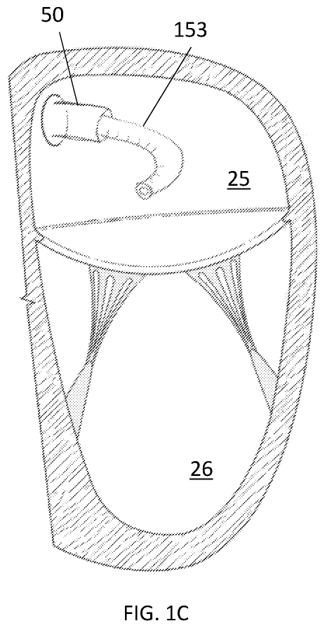

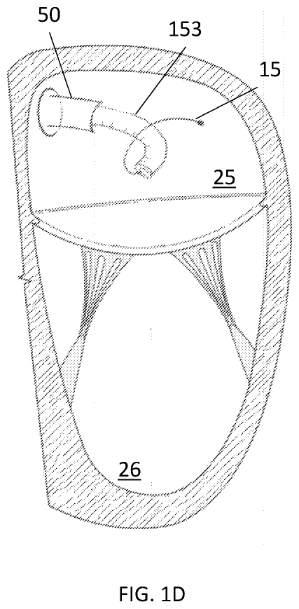

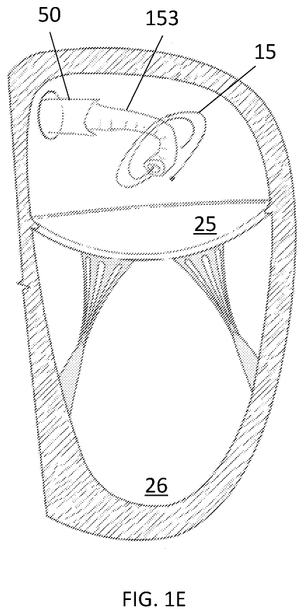

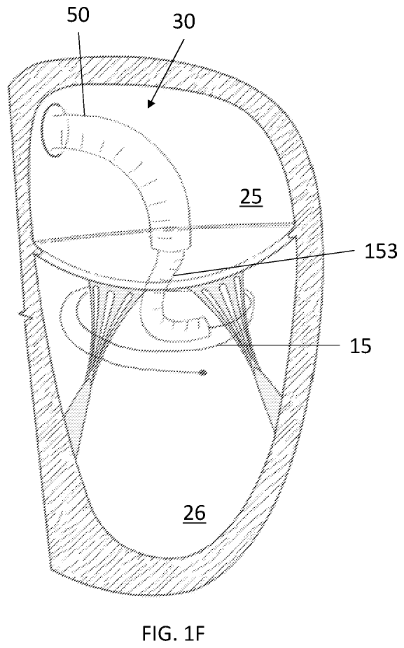

FIGS. 1A-1G show a method of delivering a spiral anchor 15 of a valve system. At FIG. 1A, a transseptal puncture is made. A guidewire 54 is then routed through the puncture site and left either in the left atrium 25 or across the mitral valve into the left ventricle 26. At FIG. 1B, an outer sheath 50 (optionally with an inner dilator 51) is tracked over the guidewire 54 until the distal end of the outer sheath 50 protrudes into the left atrium 25. The guidewire 54 and inner dilator 51 are then removed from the outer sheath 50. At FIG. 1C, an inner sheath and attached anchor guide 153 are inserted through the outer sheath 50 until the distal tip of the anchor guide 153 extends into the left atrium 25. The anchor guide 153 can be positioned and/or oriented as desired by steering the distal end of the sheath 50 and/or rotating the inner shaft and anchor guide 153 within the outer sheath 50. At FIG. 1D, once the anchor guide 153 is in the correct orientation, the anchor 15 can be pushed out through distal tip of the anchor guide 153. At FIG. 1E, the curvature of the anchor guide 153 can cause torsion on the anchor 15, causing the anchor 15 to deploy concentrically with the outer sheath 50 into the atrium 25. At FIG. 1F, the entire anchor delivery system 30 can be pushed and steered (for example, via steering mechanisms in the outer sheath 50) towards an apex of the ventricle 26, crossing through the mitral valve. In some embodiments, counter-rotation of the anchor 15 (via counter-rotation of the inner shaft and guide 153) may aid in getting the anchor across the mitral valve without tangling. Once the anchor 15 is at the correct depth within the ventricle 26, forward rotation of the anchor 15 (via forward rotation of the inner shaft and guide 153) will allow the anchor 15 to encircle the mitral leaflets and chordae. In some embodiments, the anchor 15 can be deployed towards the apex to avoid interference with mitral leaflet motion. The outer sheath 50, inner sheath, and anchor guide 153 are removed, leaving a tether 78 in place (and still attached to the anchor 15).

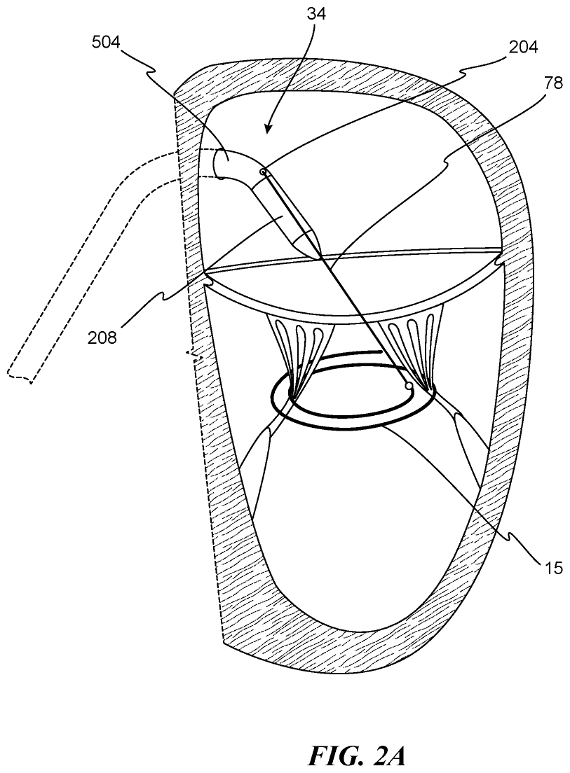

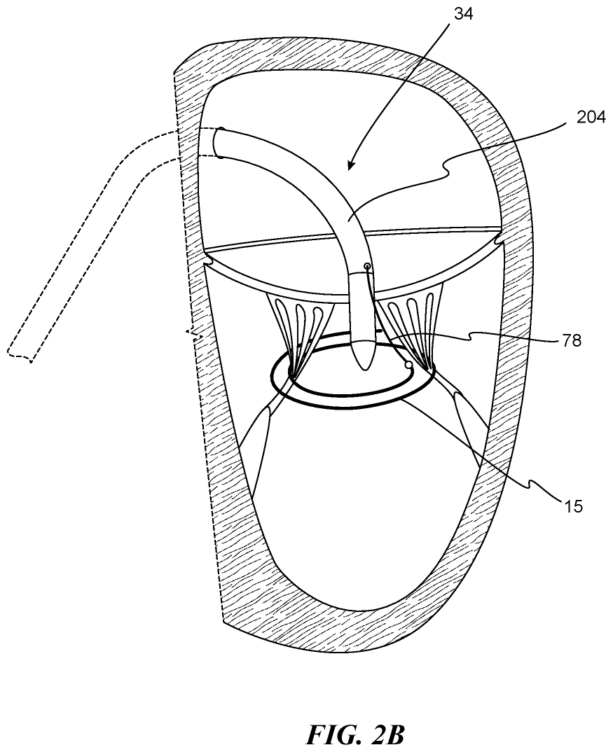

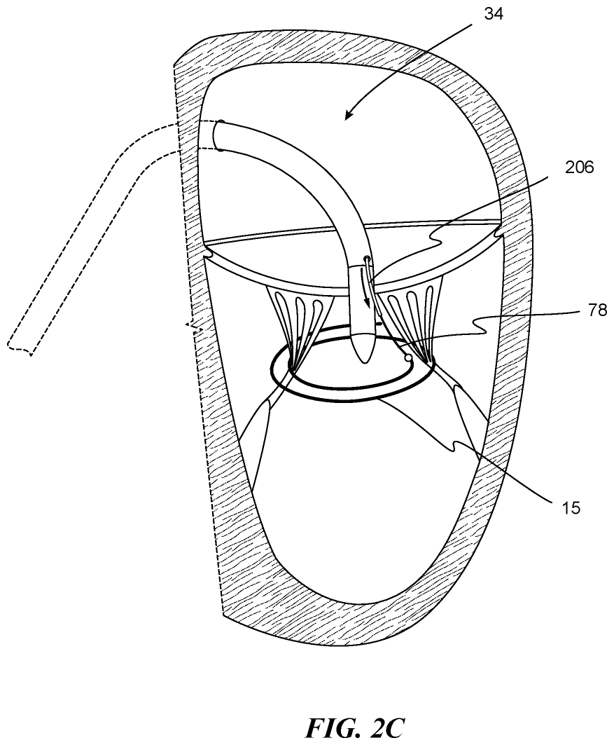

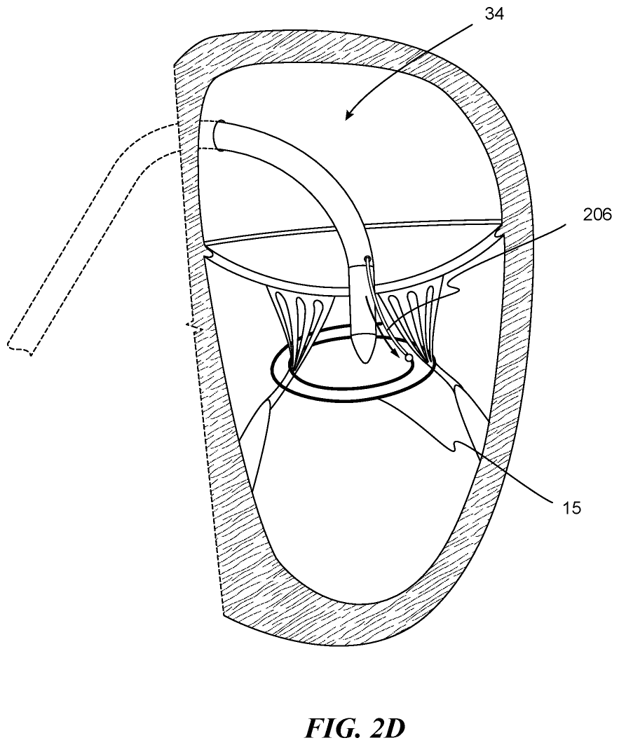

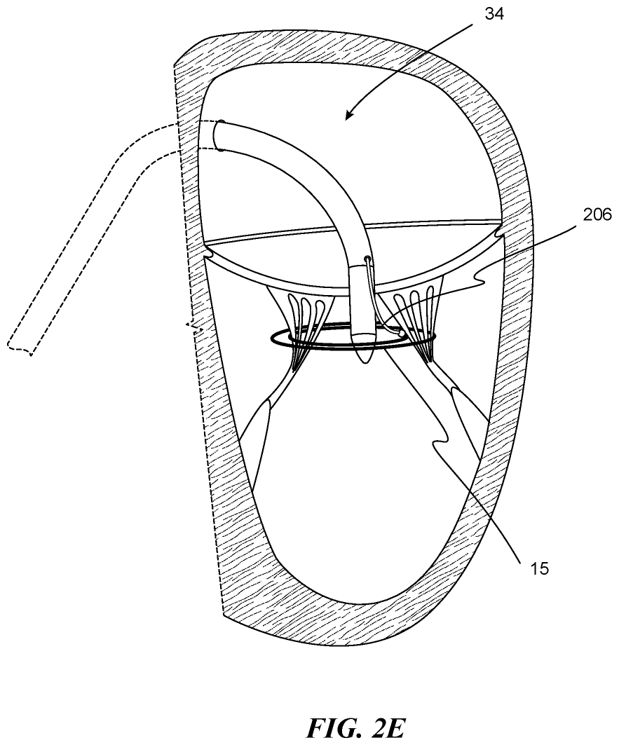

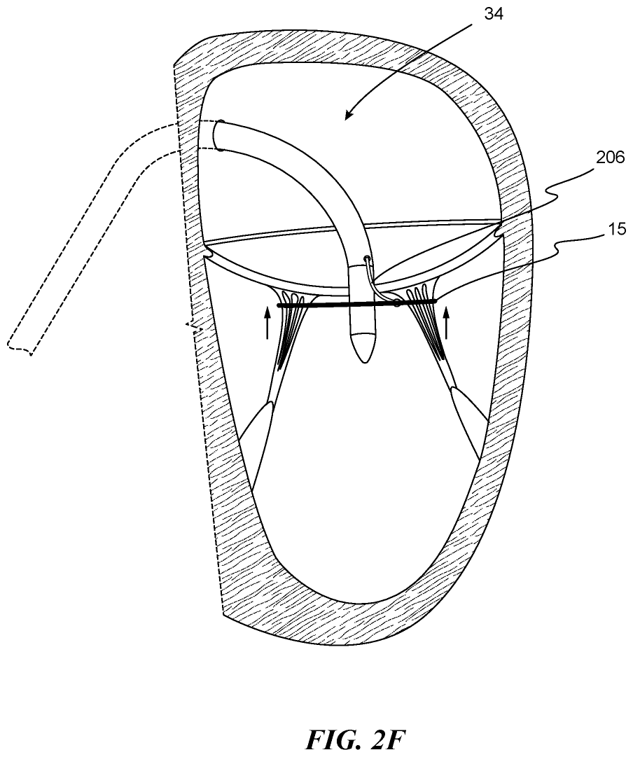

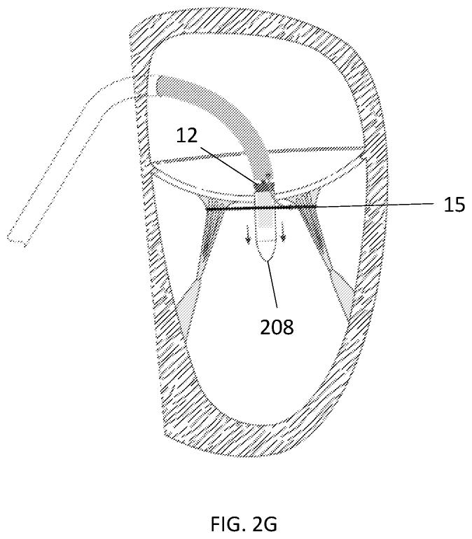

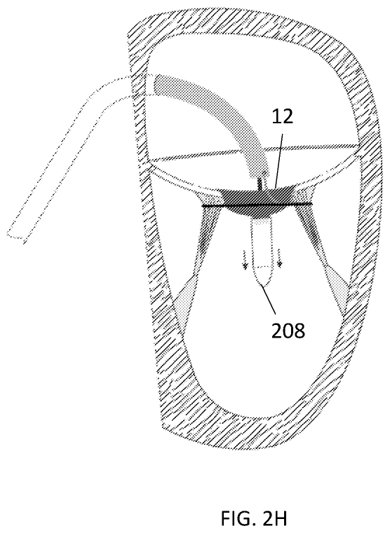

FIGS. 2A-J show an exemplary method of delivering a valve within the anchor 15 after an anchor 15 and tether 78 have already been placed (e.g., after the steps shown in FIGS. 1A-1G).

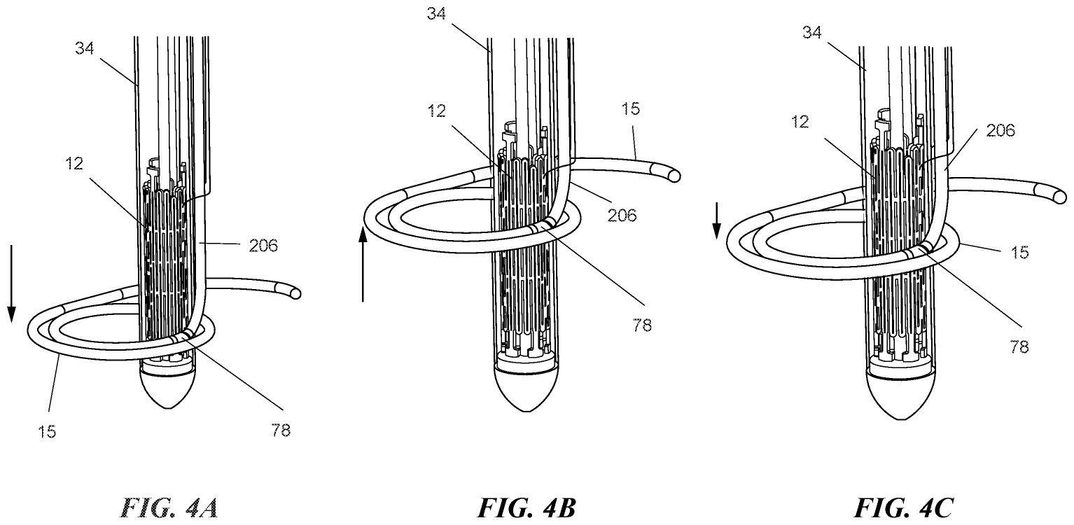

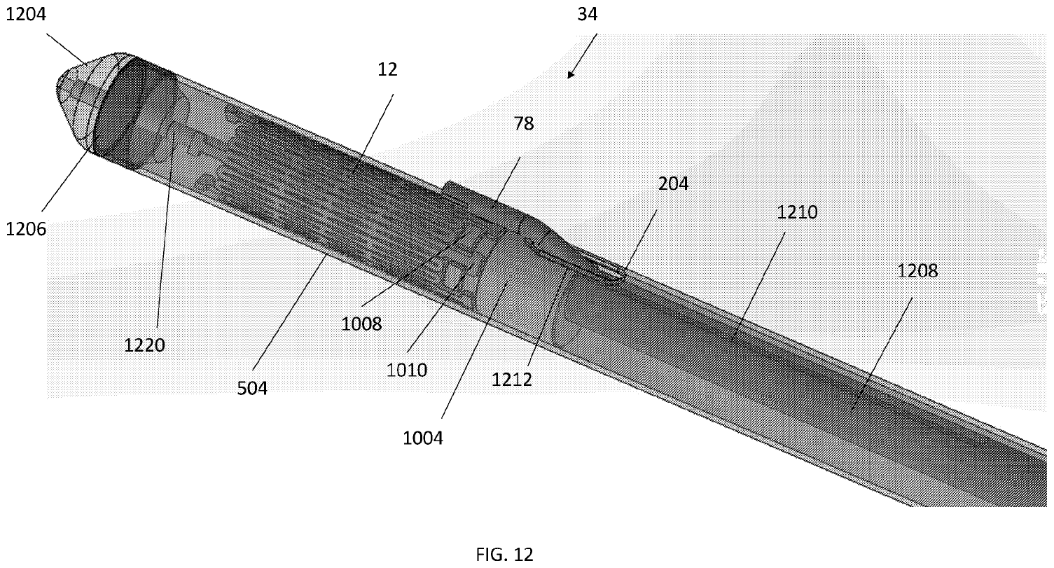

As shown in FIG. 2A, the valve delivery catheter 34 (including an outer shaft 504 and valve capsule 208) can be tracked over the tether 78 (and/or a separate guidewire) and into the left atrium. The tether 78 can be positioned within a separate lumen within the valve delivery catheter 34 and exit through a side port 204, as shown in FIGS. 2A and 2B. Other embodiments for carrying the tether 78 (e.g., monorail shown in FIGS. 4A-6L) or valve (e.g., the catheter with valve retainer shown in FIG. 12) are also possible.

As shown in FIG. 2B, the valve delivery catheter 34 can further be pushed across the native mitral valve annulus into the ventricle.

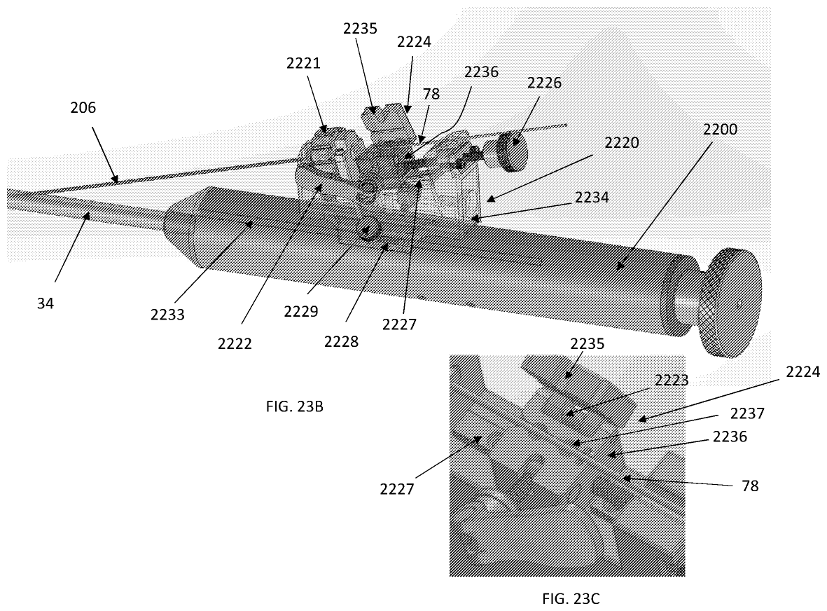

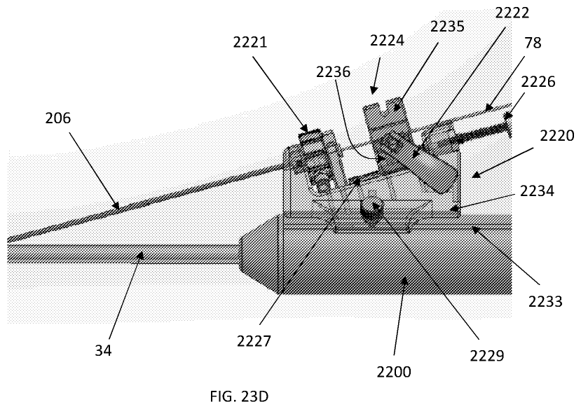

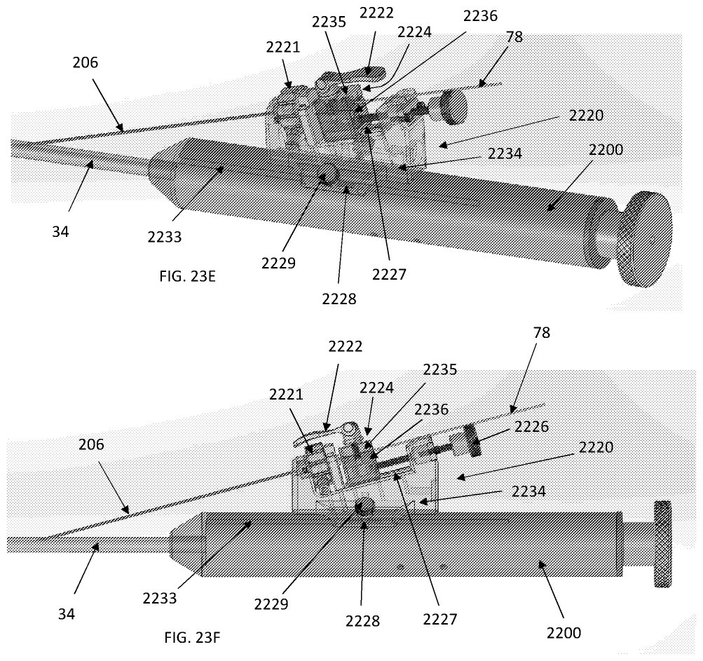

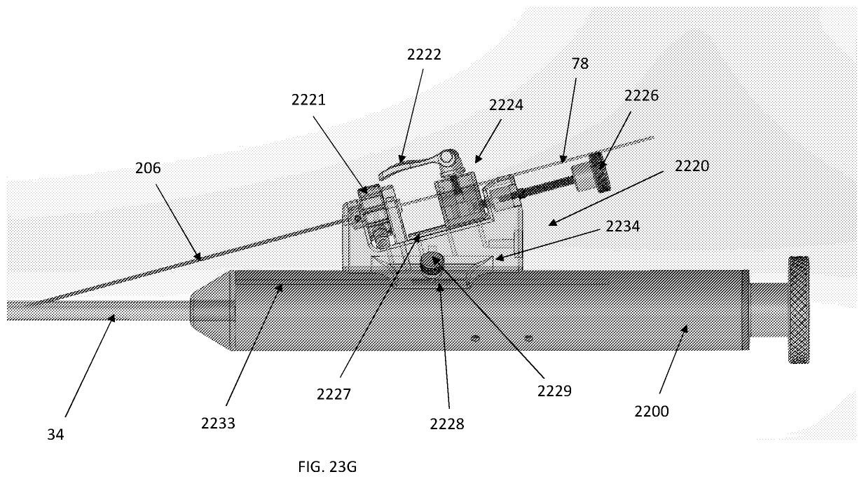

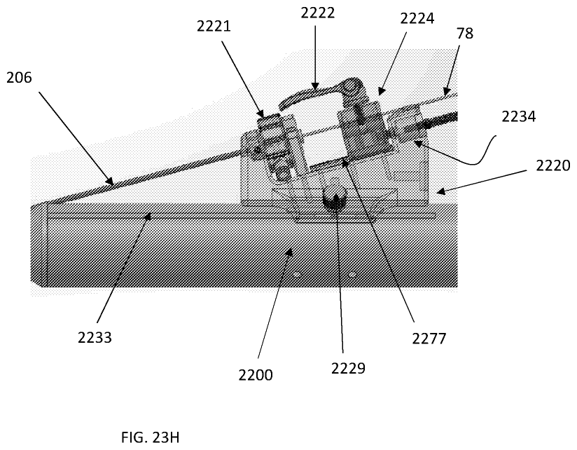

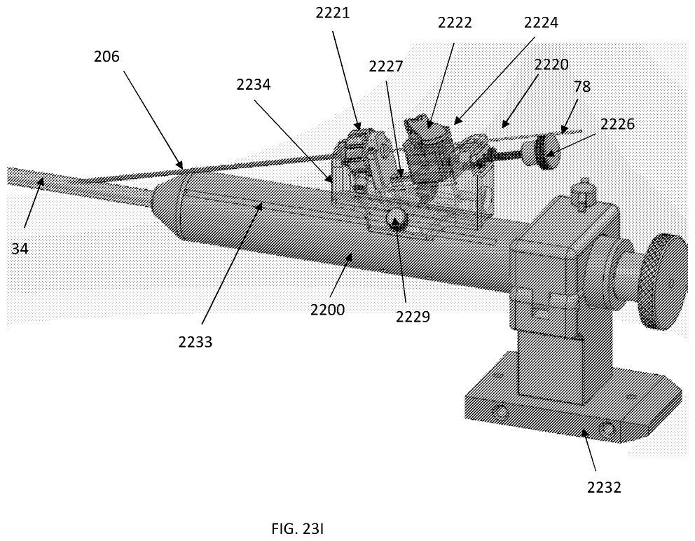

As shown in FIG. 2C, a positioning tool 206 can additionally be tracked over the tether 78. The positioning tool 206 can be tracked over the tether 78 after the valve delivery catheter 34 has been tracked thereover (as shown with respect to FIGS. 2A-2C) or simultaneously with the valve delivery catheter (e.g., as shown with respect to FIGS. 23A-23I).

As shown in FIG. 2D, the positioning tool 206 can be advanced distally over the tether 78 until a distal end of the positioning tool 206 meets a proximal end of the anchor 15 (and/or a distal tether engagement element of the tether as described further herein). The positioning tool 206 can be configured to stiffen upon engagement with the anchor or the distal tether engagement element. In some embodiments, the positioning tool 206 can stiffen with a bend that is within a plane of the spiral anchor 15. In some embodiments, the stiffened positioning tool 206 can be used to achieve axial alignment between the anchor 15 and the valve delivery catheter 34 (i.e., such that the anchor 15 sits in a plane that is perpendicular to the plane of the distal end of the valve delivery catheter 34). This adjustment can advantageously be made without reducing the encircling of the anchor 15 bout the native anatomy (e.g., chordae and leaflets).

As shown in FIG. 2E, the stiffened positioning tool 206 can additionally be used to adjust the axial alignment of the anchor 15. For example, the stiffened positioning tool 206 can be configured to adjust an axial position of the anchor 15 relative to the valve delivery catheter 34 (and/or a valve within the valve delivery catheter 34). As another example, the positioning tool 206 can pull the anchor 15 up (proximally) towards mitral annulus to better engage with mitral leaflets and reduce leakage around valve/anchor (paravalvular leakage). These adjustments can again advantageously be made without reducing the encircling of the anchor 15 bout the native anatomy (e.g., chordae and leaflets).

FIG. 2F shows the anchor 15 in position near the mitral annulus. The bent stiffened positioning tool 206 can help enable the anchor 15 to achieve planarity with the mitral annulus (e.g., if the valve delivery catheter 34 is orthogonal to the annulus). Proper positioning of the anchor 15 can additionally help ensure planar positioning of the valve frame with respect to the mitral annulus.

As shown in FIG. 2G, once the anchor 15 is properly positioned, the valve capsule 208 can be translated distally, exposing the valve frame 12.

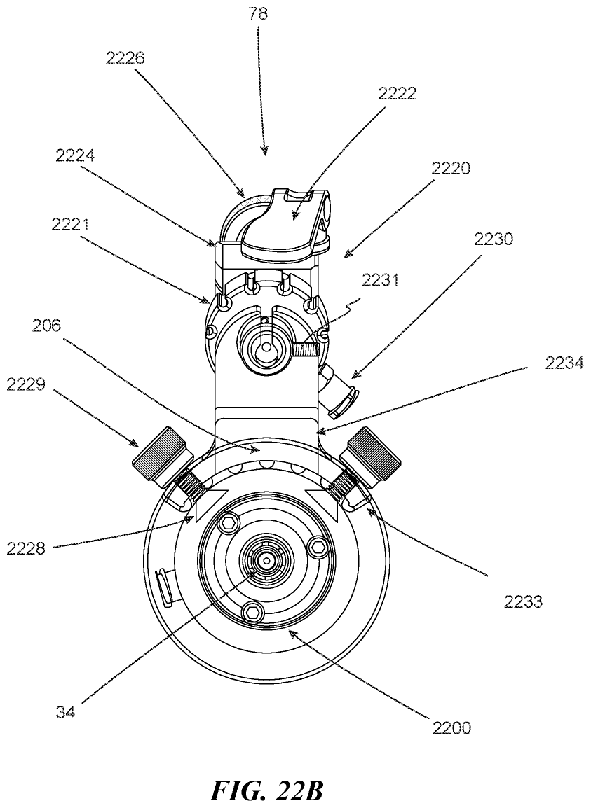

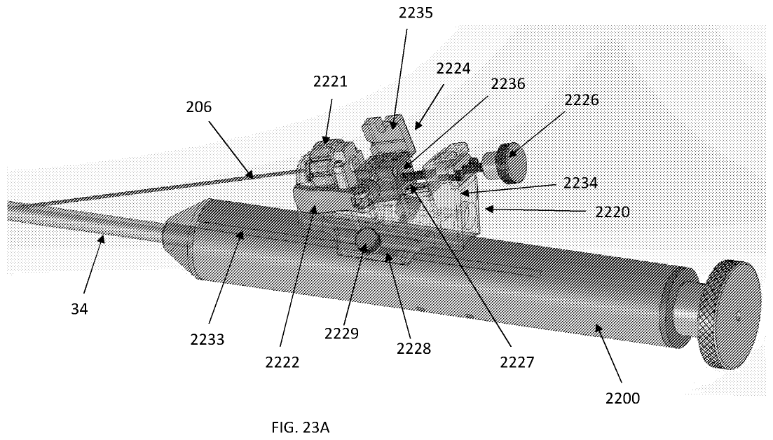

As shown in FIG. 2H, the valve capsule 208 can be further translated distally, exposing more of valve frame 12. As the valve frame 12 is exposed, the exposed portions may begin to expand, as depicted in FIG. 2H. Proximal controls (e.g., as described with respect to FIGS. 22A-23I) can help maintain the axial relationship between the valve, anchor, and anatomy. In some embodiments, a steering mechanism on the valve delivery catheter 34 can be used to help maintain the proper orientation of the valve delivery catheter 34.

As shown in FIG. 2I, the valve capsule 208 can be further translated distally until the entire valve frame 12 is exposed. Once deployed, the valve frame 12 can be held in place in the anatomy using a compression fit between the valve frame 12 and the anchor 15. The valve frame 12 may comprise a ventricular flare 160 and an atrial flare 157 with a waist positioned around a midsection of the valve frame 12. The valve frame 12 may seat against the anchor 15 such that the anchor surrounds the waist section of the valve frame 12.

As shown in FIG. 2J, the valve delivery catheter 34 and tether 78 can then be withdrawn and removed from the body, leaving the anchor 15 and valve frame 12 in place. In some embodiments, the full removal sequence includes first de-actuating (e.g., unbending or unstiffening) the positioning tool 206, releasing the tether 78 from the anchor 15, then retracting the tether 78 and the positioning tool 206 into the valve delivery catheter 34, and then retracting the valve delivery catheter 34 (with the tether 78 and positioning tool 206 therein). In other embodiments, the full removal sequence includes first de-actuating the position tool 206, then retracting the positioning tool 206 into the valve delivery catheter 34, then releasing the tether 78 from the anchor 15, and finally retracting the valve delivery catheter 34.

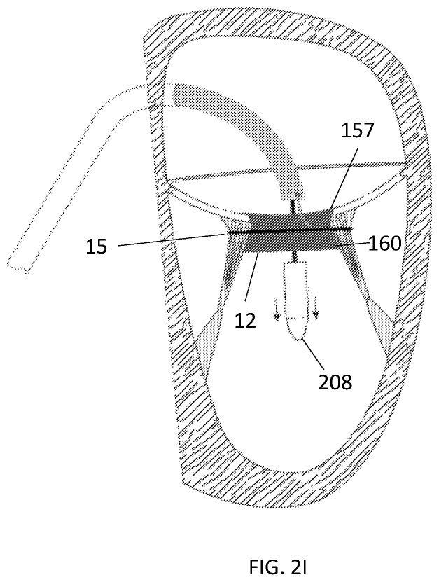

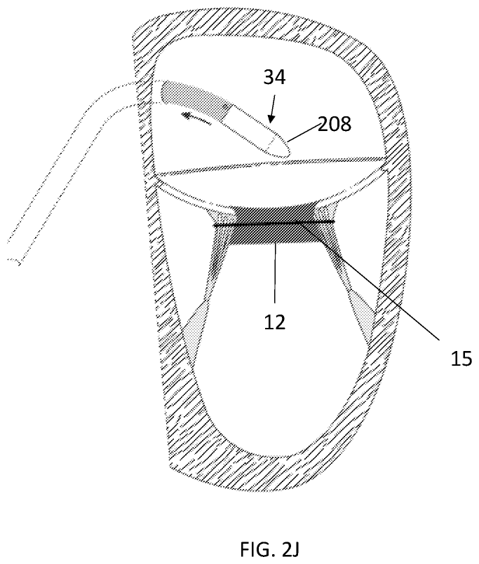

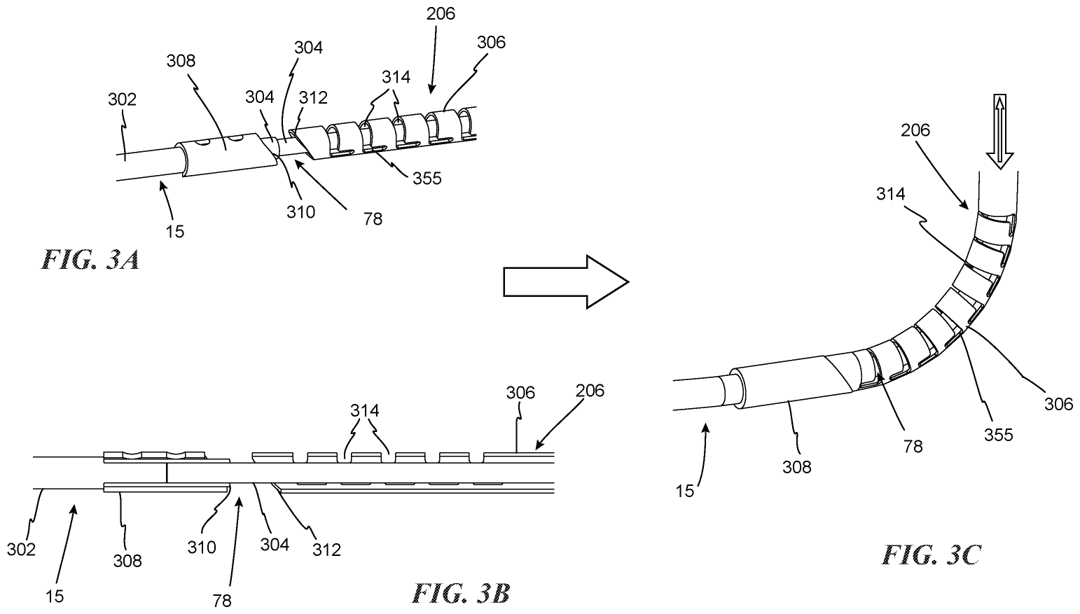

FIGS. 3A-3C show the interaction between a distal end 304 of a tether 78, a proximal end 302 of an anchor 15, and a distal end 306 of a positioning tool 206. As shown in FIG. 3A, the proximal end 302 of the anchor 15 can be attached to the distal end 304 of the tether 78 (e.g., via an attachment or release mechanism). The distal end 304 of the tether 78 can include a distal engagement element 308 attached (e.g., welded) to it. The distal engagement element 308 can include an angled or beveled proximal surface 310 generally shaped to interact or mate with an angled or beveled surface 312 of the distal end 306 of the positioning tool 206. It will be appreciated that other shapes for the proximal surface 310 of the tether and the beveled surface 312 of the positioning tool 206 are also possible, as long as the ends can be shaped to interact with one another. The interaction between the proximal surface 310 of the tether and the beveled surface 312 of the positioning tool 206 can create an aligned, torsionally stiff connection between the tether 78 (and attached anchor 15) and the positioning tool 206.

In use of the positioning tool 206, the positioning tool 206 can be advanced distally over the tether 78 until the distal end 306 of the positioning tool 206 encounters the distal engagement element 308. Further advancing the positioning tool 206 against the distal engagement element 308 can compress the distal end 306 of the positioning tool 206, thereby bending and/or stiffening the positioning tool 206. That is, as shown in FIG. 3C, the distal end 306 of the positioning tool 206 can comprise cutouts 314 (e.g., laser-cut cutouts) along a first lateral portion thereof and an axially extending spine 355 (i.e., with no cutouts) on the opposite lateral side. Compression of the distal end 306 of the positioning tool 206 can cause the distal end 306 to preferentially bend towards the cut-outs 315 (and away from the spine 355). The stiffened positioning tool 206 can be stiffened with respect to torsion and/or (e.g., further) bending of the body of the positioning tool 206. In some embodiments, the bend of the positioning tool 206 can have a maximum fixed angle (when fully compressed) of approximately 90 degrees.

As shown best in FIGS. 4A-4C, in some embodiments, the positioning tool 206 can be oriented or clocked relative to the anchor 15 (via the engagement of the surfaces 310/312) such that the bend (e.g., when at the maximum angle) is perpendicular to the plane of the anchor 15. Additionally, the bend can cause the distal end 304 of the tether 78 to be positioned in-line with (i.e., within) the plane of the spiral anchor 15. Similar to as described with respect to FIGS. 2A-2J, the positioning tool 206, once activated (i.e., stiffened and/or bent), can be used to manipulate the plane of the anchor 15 relative to the valve delivery catheter 34 (with valve 12 therein). For example, FIG. 4A shows the positioning tool 206 (and tether 78) being pushed distally, which, in turn, pushes the anchor 15 distally. FIG. 4B shows the tether positioning tool 206 (and tether 78) being pulled proximally, which, in turn, pulls the plane of the anchor 15 proximally. FIG. 4C shows the tether positioning tool 206 (and tether 78) being pushed slightly distally, such that the plane of the anchor 15 is located between the positions shown in FIGS. 4A and 4B. The position of the anchor 15 relative to the valve 12 in FIG. 4C may advantageously ensure that the waist of the frame 12 is aligned with the anchor 15 upon release.

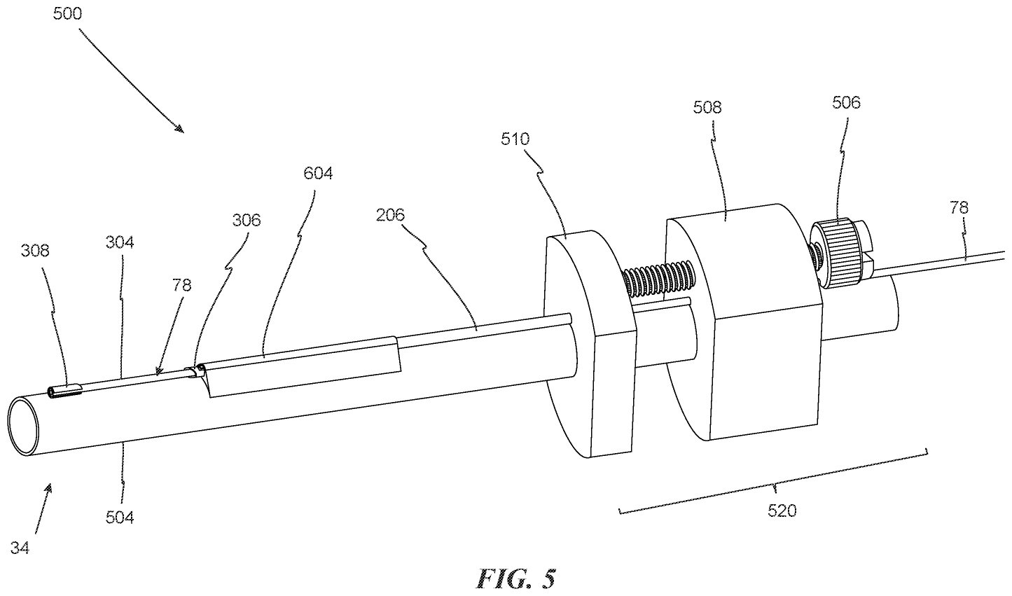

FIG. 5 shows an embodiment of an exemplary valve delivery catheter system 500 that includes a positioning tool control 520. The positioning tool control 520 can be slidably engaged with the outer shaft 504 of the delivery catheter 34. It should be appreciated that FIG. 5 is not shown to scale (e.g., the outer shaft 504, tether 78, and positioning tool 206 can include working lengths appropriate to the treatment site). Further, the positioning tool control 520 can include a tether tensioning screw 506, a tether latching block 508 configured to clamp the tether 78 within the positioning tool control 520, and a positioning tool block 510 that is fixedly attached to the positioning tool 206. In some embodiments, and as shown in FIG. 5, the tether 78 and positioning tool 206 can be configured to be carried on the outer shaft 504 in a monorail fashion, e.g., through an exterior monorail channel 604 (though the tether can also pass through a lumen of the outer shaft 504, as described with respect to FIGS. 2A-2J). The system 500 can further include a control (e.g., a handle, such as handle 1300) configured to control deployment of the valve from the valve delivery catheter 34.

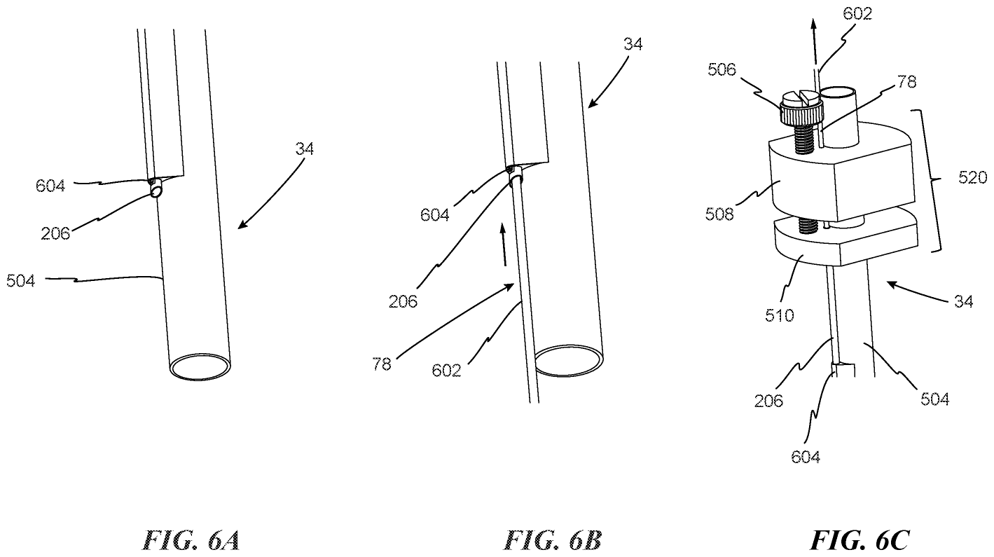

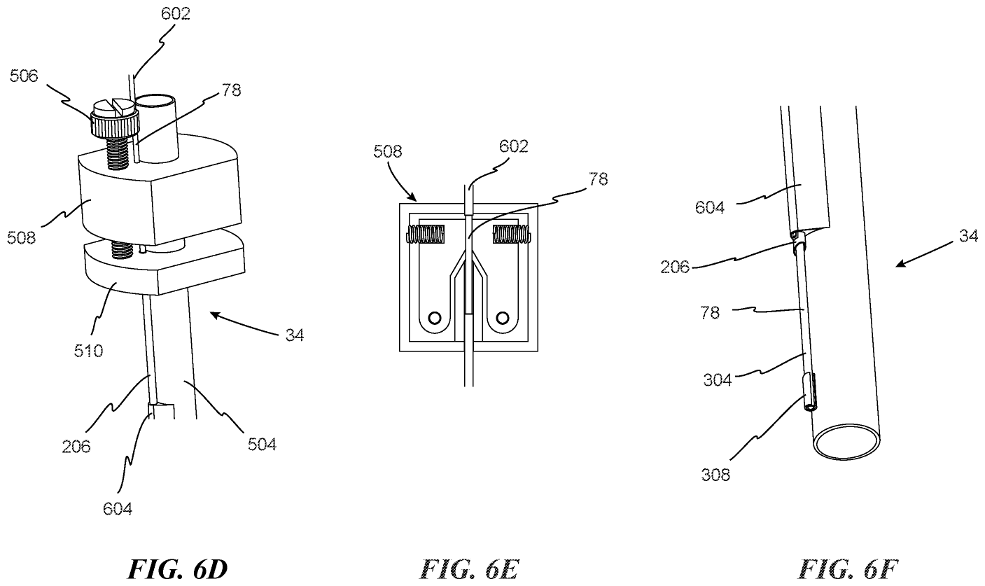

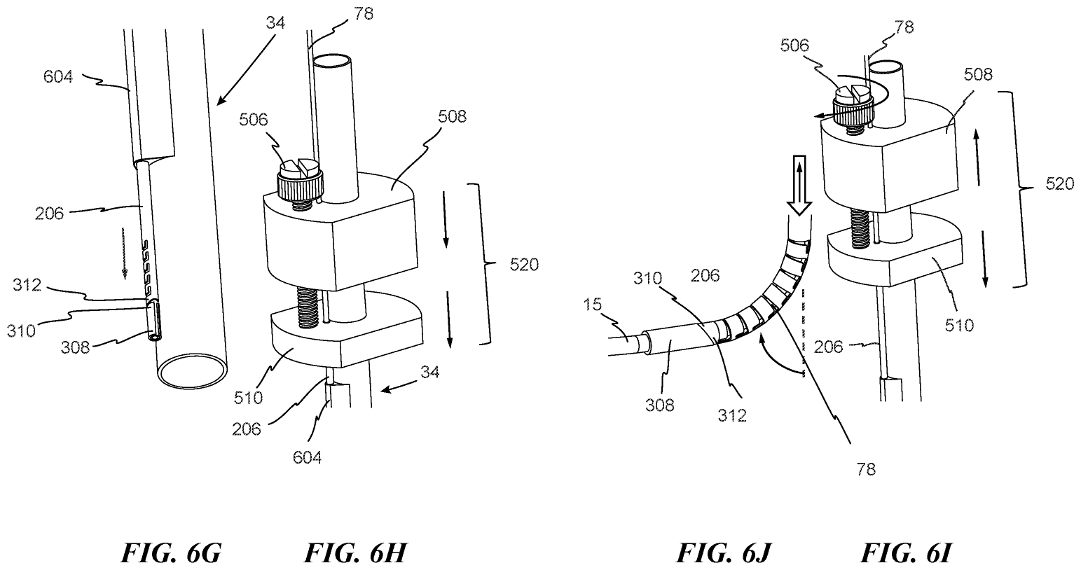

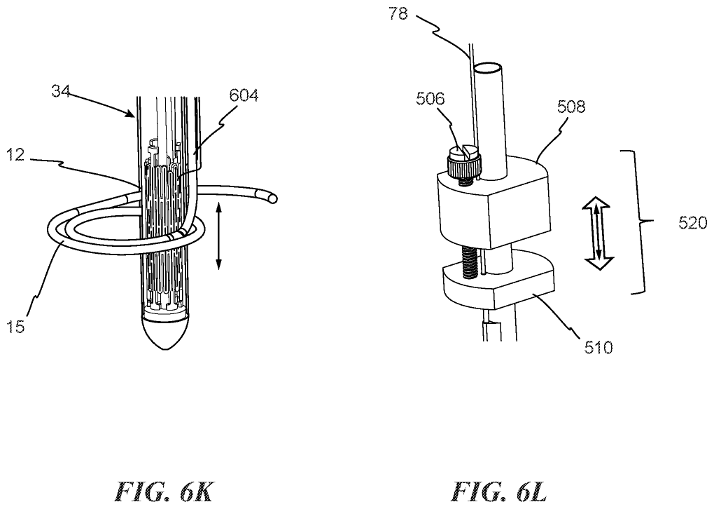

FIGS. 6A-6L show an exemplary method of using the system 500 with a pre-deployed tether 78 (i.e., a tether 78 that is attached to an anchor 15 within a native valve) to adjust a relative position of the anchor 15 and a valve 12 that is positioned within the valve delivery catheter 34. Referring to FIG. 6A, the system 500 can include a positioning tool 206 positioned (e.g., pre-installed) in the monorail channel 604 (and attached at the proximal end to the positioning tool block 510). As shown in FIG. 6B, a proximal end 602 of the tether 78 can be inserted into the distal opening of the monorail channel 604. Referring to FIG. 6C, the delivery system 500 can be tracked distally over the tether 78 such that the proximal end 602 of the tether 78 extends through the positioning tool control 520. As shown in FIG. 6D, the tether 78 can then be clamped into the positioning tool control 520 by activating the latching block 508. An interior view of an exemplary latching block 508 is shown in FIG. 6E and described in further detail below. As shown in FIG. 6F, when the tether 78 is clamped into position, the distal engagement element 208 of the tether 78 can be proximate to the distal end of the positioning tool 206. As shown in FIGS. 6G and 6H, the positioning tool 206 can then be advanced (e.g., via distal movement of the entire positioning tool control 520 relative to the valve delivery catheter 34) until the distal beveled surface 312 of the positioning tool 206 meets the beveled surface 310 of the valve engagement element 308 of the tether 78. Referring to FIGS. 6I and 6J, the tether 78 can then be pulled (or tensioned) proximally relative to the positioning tool 206 to compress the distal end 306 of the positioning tool 206 to activate the bend in the positioning tool 206 (shown in FIG. 6I). To pull the tether 78 proximally, the tether latching block 508 (holding the clamped proximal end 602 of the tether 78) can be moved proximally relative to the positioning tool block 510. The relative movement between the tether latching block 508 and the positioning tool block 510 can be actuated by turning the tether tensioning screw 506, as shown in FIG. 6J. Referring to FIGS. 6K and 6L, the axial position of the anchor 15 relative to a valve 12 within the valve delivery catheter 34 can be controlled by sliding the entire positioning tool control 520 proximally or distally (e.g., along the outer catheter shaft 504). Once the anchor 15 has been properly positioned, the valve 12 can be released from the valve delivery catheter 34 as described herein.

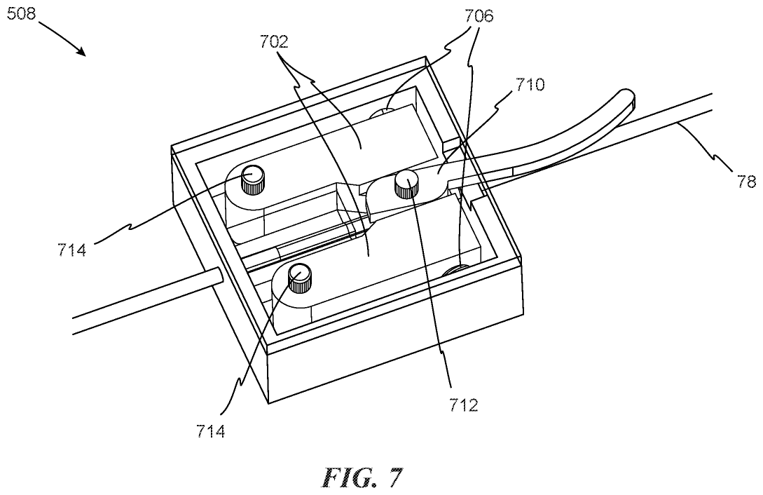

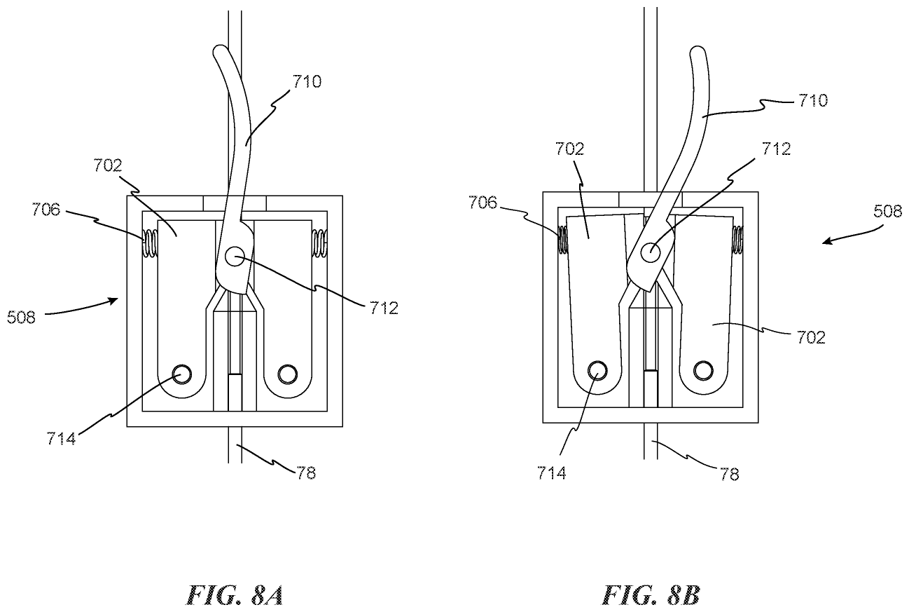

FIG. 7 shows an exemplary embodiment of the tether latching mechanism 508. The tether latching block 508 can include a pair of jaws 702, and the tether 78 can be configured to run in between the jaws 702. The jaws 702 can be fixed by and configured to rotate around hinge points 714. Springs 706 can be positioned on either side of the jaws 702 so as to bias the jaws 702 towards a closed position. In the closed position, the jaws 702 can clamp the tether 78 therebetween. The tether latching block 508 can further include a release lever 710 positioned between the free ends of the jaws 702 proximate to the tether 78. The release lever 710 can be configured, when activated, to push the jaws 702 apart, releasing the clamp on the tether 78 (for example, to release the valve delivery catheter 34 from the tether 78 for adjustment of the position of the valve delivery catheter 34 or for retraction of the valve delivery catheter 34 after deployment of the valve 12). The release lever 710 can be configured to rotate about a hinge point 712 to open the jaws 702. FIGS. 8A-8B illustrate a method of using the release lever 710 to open the jaws 702. As shown in FIG. 8A, the tether 78 can be captured between the jaws 702. Rotation of the lever 710 about hinge point 712 causes the lever 710 to cam the jaws 702 open and disengage the tether 78, as shown in FIG. 8B.

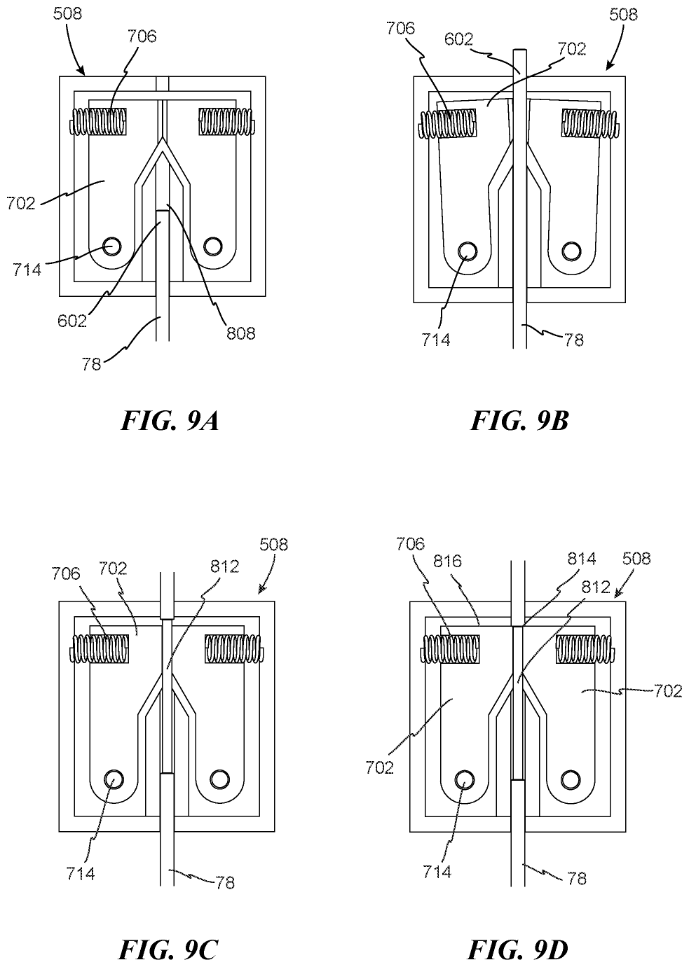

In some embodiments, the latching block 508 can be activated (e.g., clamped) automatically during tracking of the system 500 over the tether 78 when a narrowed section 812 of the tether 78 (near the proximal end 602 of the tether 78) extends between the jaws 702. FIGS. 9A-9D show an exemplary method of tracking the proximal end 602 of the tether 78 through the latching block 508 (i.e., during loading of the valve delivery catheter 34 over the tether 78) until the proximal end 602 of the tether 78 automatically activates the clamping. As shown in FIG. 9A, the proximal end 602 of the tether 78 can be tracked proximally through a lumen 808 into the latching block 508. Referring to FIG. 9B, as the tether 78 is tracked proximally, the proximal end 602 of the tether 78 can contact jaws 702, causing the jaws 702 to open and allow the tether 78 to freely slide therebetween. As shown in FIG. 9C, as the tether 78 is still further tracked proximally, a narrow section 812 of the tether 78 can enter the jaws 702, allowing the jaws 702 to pivot inward and lock the tether 78 in place. Referring to FIG. 9D, tension on the tether 78 can cause the wider diameter notch 814 of the proximal end 602 of the tether 78 to rest on the face 816 of the jaws 702.

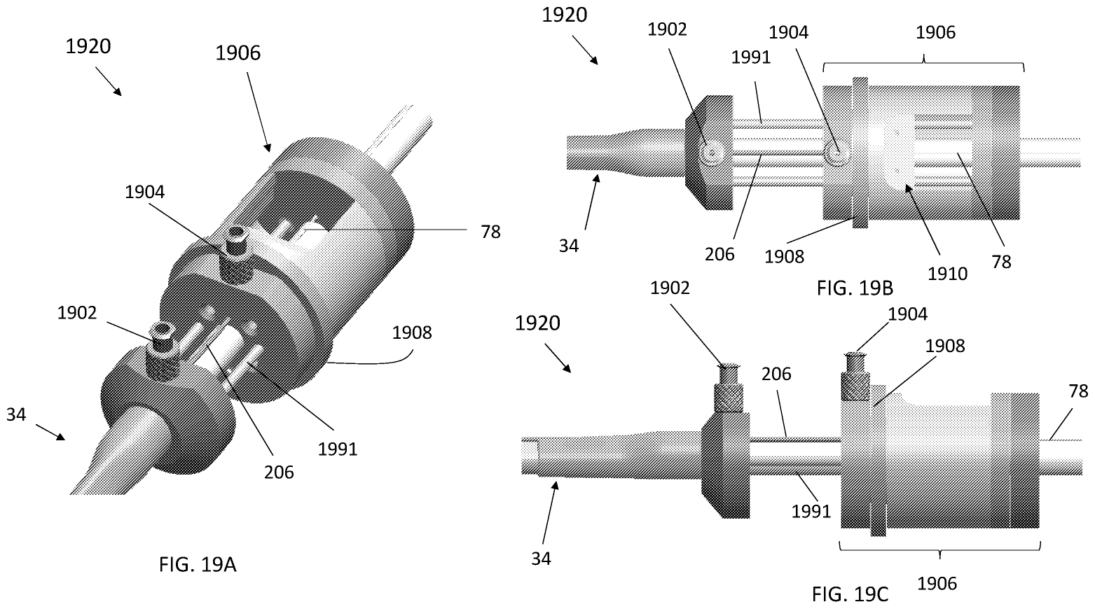

FIGS. 19A-19C show perspective, top, and side views, respectively, of an embodiment of another positioning tool control 1920 for use with a valve delivery system. The positioning tool control 1920 includes a positioning carriage 1906 configured to axially slide along rails 1991. The positioning carriage 1906 can be fixedly connected at its distal end to the positioning tool 206. Additionally, the positioning carriage 1906 can include a tether engagement mechanism 1910 and a tensioning dial 1908. The positioning tool control 1920 can further include a tether or monorail lumen flush port 1902 and a positioning tool lumen flush port 1904.

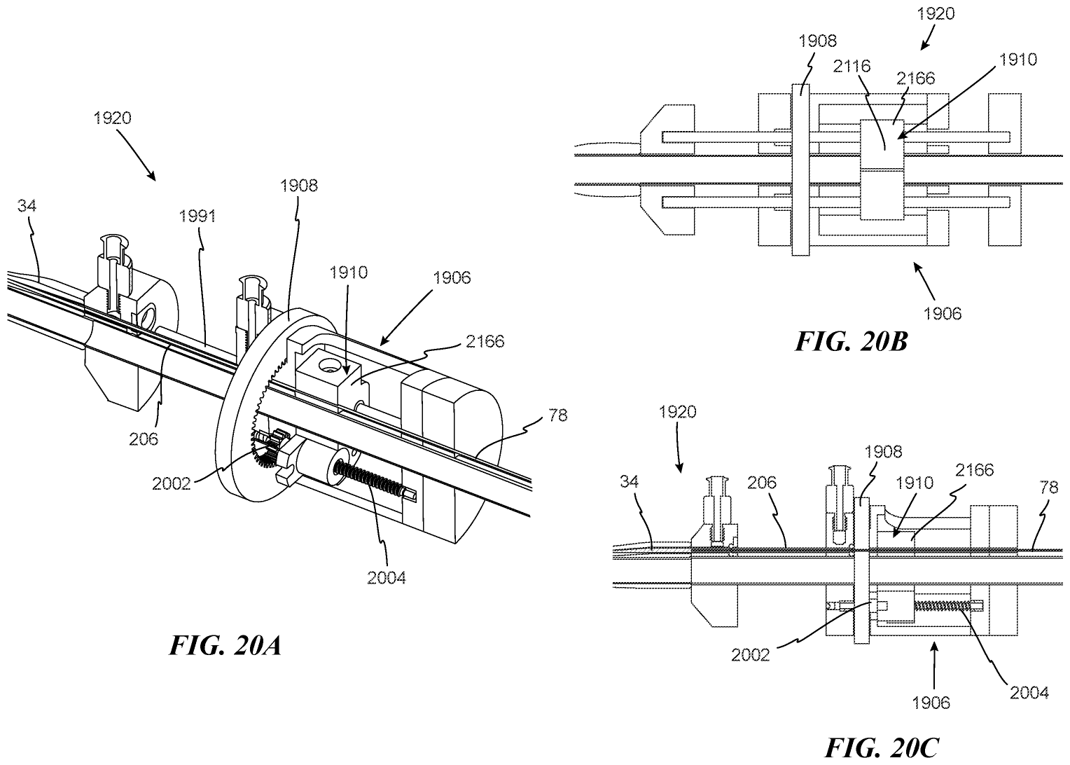

FIGS. 20A-20C show perspective, top, and side views, respectively, of the internal components of the positioning tool control 1920. The tensioning dial 1908 can include a ring gear configured to interact with a spur gear 2002. The spur gear 2002 can configured to interact with lead screw 2004. The ring gear, spur gear 2002, and lead screw 2004 can make up the drive train for the tensioning dial 1908. In some embodiments, the ring gear can include about 60 (or about 50-70) teeth. In some embodiments, the spur gear 2002 can include about 12 (or about 9-15) teeth. In some embodiments, the lead screw 2004 can be about ⅛″ and about 20.8 tpi. In some embodiments, about 3.12″ of ring gear turns (or about 2.75-3.5″) can translate to about 0.75″ (or about 0.6″ to about 0.9″) of carriage 1906 travel. As is also shown in FIGS. 20A-20C, the tether engagement mechanism 1910 can include a clamp 2166 and clamping screws 2116.

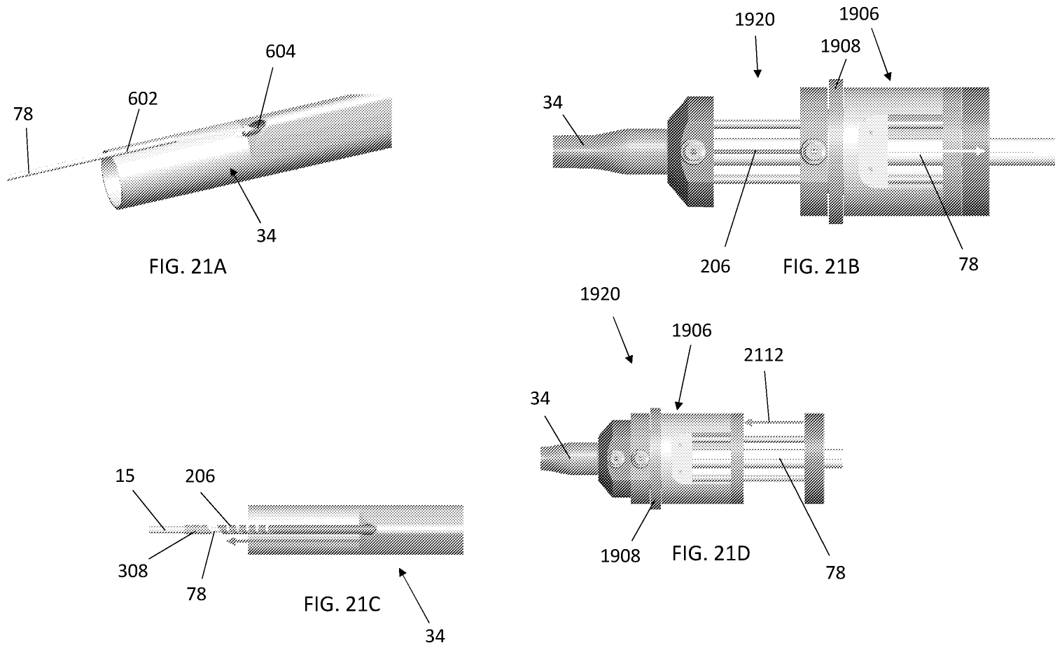

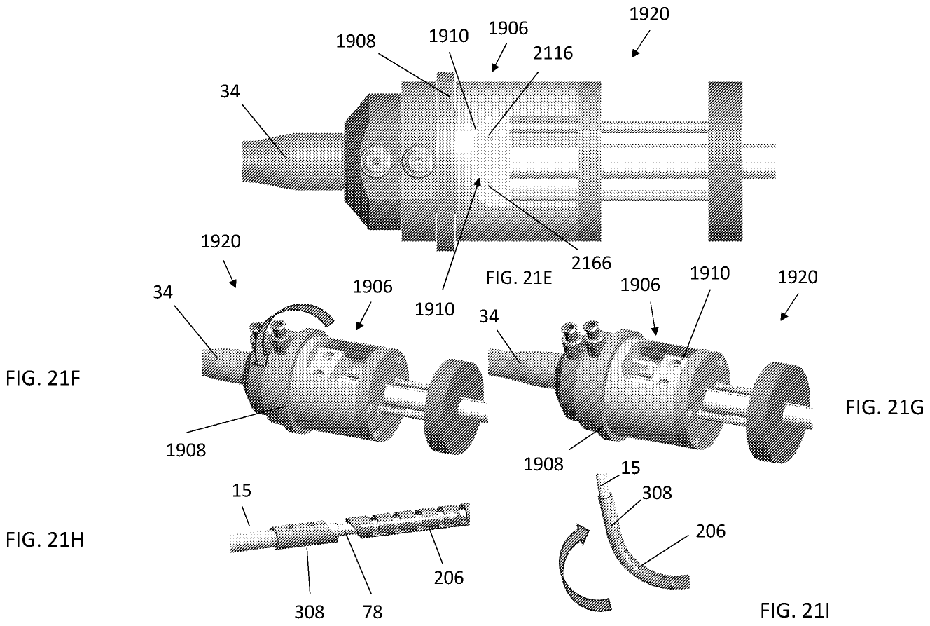

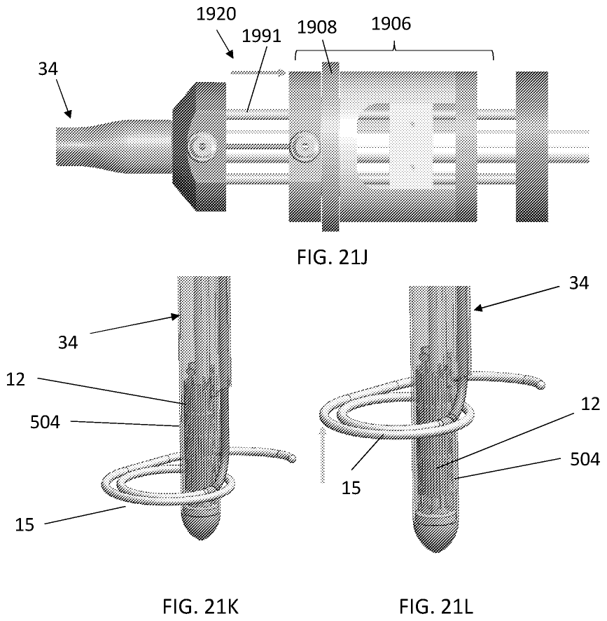

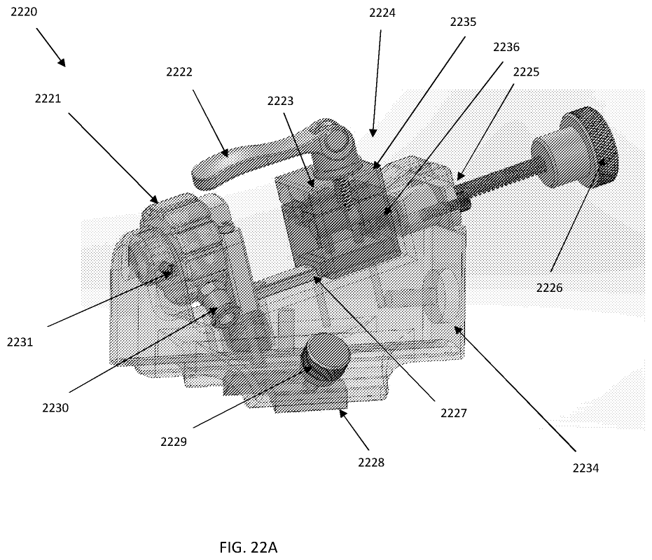

FIGS. 21A-L show an exemplary method of using the positioning tool control 1920 shown in FIGS. 19A-20C to adjust the position of an anchor 15 relative to a valve 12. Referring to FIG. 21A, the proximal end 602 of the tether 78 (which can be attached at its distal end to an anchor 15 in the heart) can be loaded into the tether channel 604. The valve delivery catheter 34 can then be loaded over the tether 78 (e.g., into the atrium of the heart) until the tether 78 reaches and travels through the positioning tool control 1920. As shown in FIG. 21B, while advancing the valve delivery catheter 34 to the atrium, the user can continue to pull the tether 78 proximally (as indicated by the arrow) through the positioning tool control 1920 to remove slack in the tether 78. Referring to FIGS. 21C and 21D, the positioning tool 206 can be deployed from the channel 604 to or towards the tether engagement element 308 (in a direction indicated by the arrow in FIG. 21C). As shown in FIG. 21D, the positioning tool 206 can be deployed distally by sliding positioning carriage 1906 distally along the rails 1991 (in the direction indicated by arrow the arrow) from the proximal position shown in FIG. 21B to the distal position shown in FIG. 21D. Referring to FIG. 21E, when tip of positioning tool 206 is near the distal engagement element 308 of the tether 78, the user can clamp down the tether engagement mechanism 1910 (i.e., to clamp the tether 78 in place relative to the carriage 1906) by tightening the clamping screws 2116 against the clamp 2166. As shown in FIG. 21G, the tensioning knob 1908 can then be rotated, causing the tether engagement mechanism 1910 to move from the distal position shown in FIG. 21F to the proximal position shown in FIG. 21G (movement of the tether engagement mechanism 1910 is shown by the arrow in FIG. 21G). Referring to FIGS. 21H and 21I, because the tether engagement mechanism 1910 is clamped to the tether 78, the proximal motion of the tether engagement mechanism 1910 will cause the tether 78 to be pulled proximally relative to the positioning tool 206 (shown by the arrow in FIG. 21H), thereby causing the positioning tool 206 to bend and/or stiffen as shown in FIG. 21I.

Referring to FIGS. 21J-21L, after the positioning tool 206 has been bent and/or stiffened, the positioning carriage 1906 (with attached positioning tool 206) can be slid axially (FIG. 21J) to fine tune anchor 15 height relative to a valve 12 positioned within the outer shaft 504 of the valve delivery catheter 34 (e.g., the anchor 15 can be moved proximally as shown from FIG. 21K to FIG. 21L).

FIGS. 22A-22B and 23A-23I show another embodiment of a positioning tool control 2220 that is configured to attach to a valve delivery handle 2200. The handle 2200 can be similar to any other valve delivery handle described herein, such as handle 1300. Further, although the proximal end of the position tool 206 is shown in FIGS. 23A-23I as extending from a port in the valve delivery catheter 34, it can also extend from a port in the handle 2200 (e.g., similar to handle 1300).

Referring to FIGS. 22A-22B, the positioning tool control 2220 can include a housing 2234 configured to support a positioning tool rotary hub 2221 (to which the positioning tool 206 can be mounted). A spring plunger 2231 on the rotary hub 2221 can be configured to engage with grooves along an external surface of the distal small diameter circumference of the rotary hub 2221 to enable indexed clocking of the orientation of the positioning tool 206 (e.g., relative to the distal engagement element of the tether 78). A flush port 2230 on the rotary hub 2221 can enable flushing of the lumen of the positioning tool 206. Further, the housing 2234 can include a pair of dovetail tracking nuts 2228 that are configured to slide within a dovetail track 2233 on the handle 2200. A pair of adjustment knobs 2229 can be configured to tighten to fix the position of the housing 2234 relative to the handle 2200 or loosen to enable sliding of the housing 2234 relative to the handle 2200.

The positioning tool control 2220 can further include a tether clamping assembly 2224 that is configured to slide relative to the housing 2234 along a linear slide assembly 2227. The tether clamping assembly 2224 can include a clamp top 2235 pivotably attached to a clamp bottom 2236 via a clamp torsion spring 2223. Further, the clamp bottom 2236 can include a groove 2237 (see FIG. 23C) configured to house a tether 78 therein. The tether clamping assembly 2224 can be locked and tightened via pivoting and rotation of a tether cam lock 2222. Further, movement of the tether clamping assembly 2224 along the linear slide assembly 2227 can be controlled via a linear adjustment knob 2226 and leadscrew and nut assembly 2225.

FIGS. 23A-23I show an exemplary method of using the positioning tool control 2220 shown in FIGS. 22A-22B to position an anchor relative to a valve. Referring to FIG. 23A, the positioning tool control 2220 can be preloaded or attached to the handle 2200 of the valve delivery catheter 34. The clamp top 2235 can be opened to enable the tether clamping assembly 2224 to receive a tether therein. Referring to FIGS. 23B and 23C, the valve delivery catheter 34 (with valve therein) and positioning tool 206 can be simultaneously tracked distally over a tether 78 (i.e., a tether 78 that is connected to a spiral anchor positioned in the heart) until the distal end of the positioning tool 206 is positioned proximate to the distal engagement element of the tether 78. The proximal end of the tether 78 can be placed in the groove 2237 within the tether clamping assembly 2224. Referring to FIG. 23D, the housing 2234 can then be moved distally along the tracks 2233 in the handle 2200 so as to move the positioning tool 206 relative to both the tether 78 and the valve delivery catheter 34 until the distal end of the positioning tool 206 is engaged with the distal engagement element of the tether 78 (as described herein). If necessary, the rotary hub 2221 can be rotated to enable proper orientation of the distal end of the positioning tool 206 and the distal engagement element of the tether 78. Referring to FIG. 23E, once the distal end of the positioning tool 206 and the distal engagement element of the tether 78 are engaged, the housing 2234 can be fixed in position relative to the handle 2200 by rotating the adjustment knobs 2229. During this step, the tether 78 can also be clamped within the tether clamping assembly 2224 by closing the clamp top 2235 and pivoting the tether cam lock 2222 from the open position (shown in FIGS. 23A-23D) to the closed or engaged position shown in FIG. 23E. Referring to FIG. 23F, the tether cam lock 2222 can further be twisted or rotated to increase the clamping force on the tether 78. Referring to FIG. 23G, the tether clamping assembly 2224 can then be moved proximally along the linear slide assembly 2227 (by rotating the adjustment knob 2226) so as to tension the tether 78 relative to the positioning tool 206 to cause the positioning tool 206 to actuate or bend (as described herein). Referring to FIG. 23H, the position of the anchor relative to the valve (positioned within the valve delivery catheter 34) can then be adjusted by sliding the positioning tool control 2220 proximally along the tracks 2233, thereby pulling the positioning tool 206 and the tether 78 (and anchor) proximally (e.g., against the mitral valve annulus). In some embodiments, the positioning tool control 2220 and handle 2200 can be moved together (e.g., proximally) to adjust a position of both the anchor and the valve. For example, as shown in FIG. 23I, the handle 2200 can be clamped to a carriage 2232 that is configured to slide along a rail or tracks. Once the anchor and valve are in the desired position, the valve can be released from the valve delivery catheter 34 to an expanded position within the anchor.

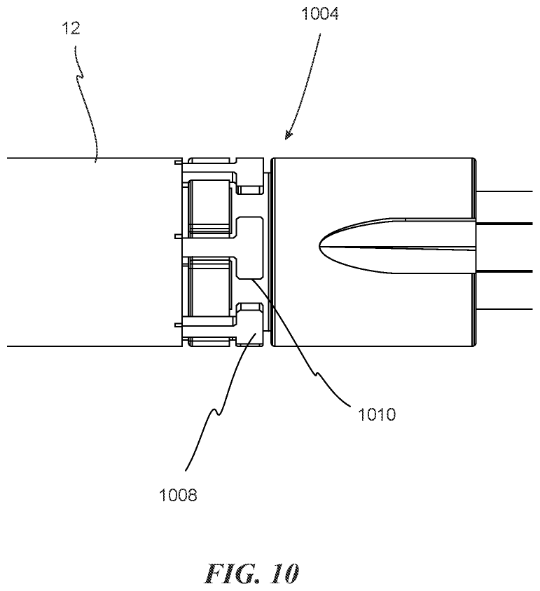

In some embodiments, the valve frame 12 can be held in place with respect to the valve delivery catheter 34 with a valve retainer 1004. FIG. 10 shows an embodiment of a proximal end of the valve frame 12 attached to an exemplary valve retainer 1004. A proximal end of the valve frame may include tabs 1008 (e.g., T-shaped tabs) configured to interact with corresponding cutouts 1010 in a distal end of the valve retainer 1004. The interaction between the tabs 1008 and the cutouts 1010 may be through an interference fit. It will be appreciated that, in some embodiments, the valve retainer 1004 may include tabs configured to interact with corresponding cutouts on a proximal end of the valve frame 12. It will also be appreciated that the tabs 1008 may include other shapes as long as the shape is selected to provide a desired fit with corresponding cutouts.

FIG. 12 depicts an exemplary valve delivery catheter 34 including the valve retainer 1004 positioned within the outer shaft 504 and attached to the valve frame 12 such that the cutouts 1010 mate with the tabs 1008 on the valve frame 12. The valve delivery catheter 34 further includes an atraumatic distal nosecone 1204 attached to a central nosecone shaft 1220 running centrally through the valve delivery catheter 34. In some embodiments, the nosecone shaft 1220 can include a lumen therein configured to pass a guidewire therethrough. The outer shaft 504 can be configured to move proximally relative to the nosecone 1204 and nosecone shaft 1220 to expose the valve frame 12 and valve retainer 1004. In some embodiments, the nosecone 1204 can include a marker 1206 (e.g., a radiopaque marker) just proximal thereto.

As shown in FIG. 12, the delivery catheter 34 can further include a distal side port 204 in the outer shaft 504 configured to pass the tether 78 therethrough. In this embodiment of the delivery catheter 34, the tether lumen extends within a tether shaft 1208 within the outer shaft 504 (i.e., rather than within a monorail lumen). The tether shaft 1208 can, in some embodiments, be a dual lumen shaft (e.g., for access of the tether 78 and a guidewire). The tether shaft 1208 can further include a longitudinal notch 1210 extending axially along a lateral side of the tether shaft 1208. The longitudinal notch 1210 can be configured to align with the side port 204 (and, correspondingly, with a groove 1212 in the proximal end of the valve retainer 1004). The tether notch 1210 can enable the tether 78 to remain fixed axially relative to the valve delivery catheter 34 (and track out through the distal side port 204) as the outer shaft 504 is pulled proximally to expose the valve frame 12 and retainer 1004. The tether shaft 1208 can, in some embodiments, be offset from the center of the outer shaft 504, which can provide enhanced engagement of the tether 78 with the side port 204.

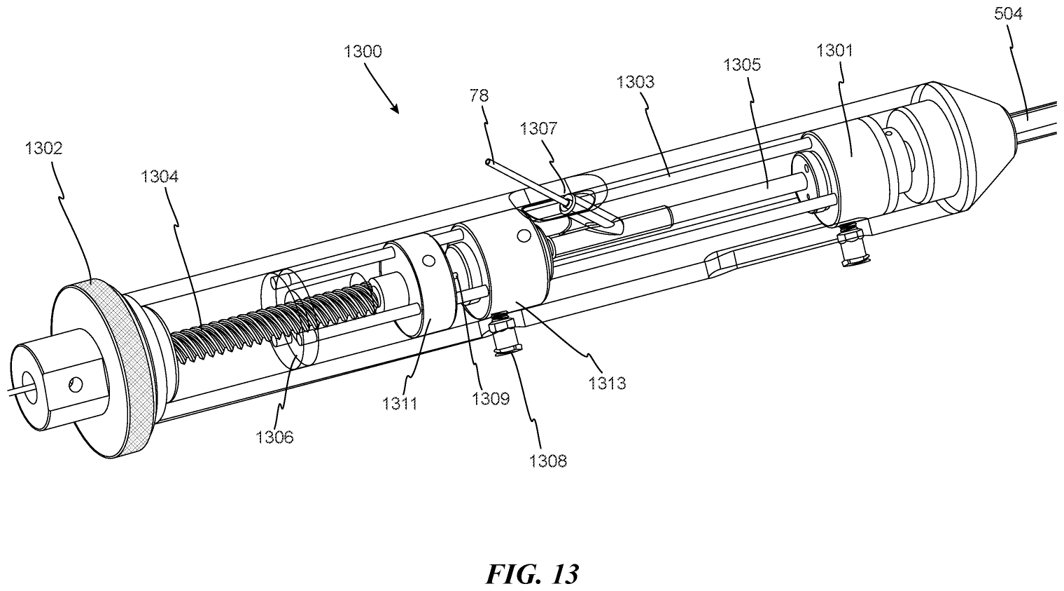

FIG. 13 shows an embodiment of a handle 1300 of a valve delivery device configured to control the valve delivery catheter 34 of FIG. 12. The handle 1300 is attached to the outer shaft 504 via a hub 1301. The hub 1301, in turn, is connected back to a nut 1306 via rods 1303. The nut 1306 is configured to move along a threaded bolt 1304 via activation (rotation) of the deployment knob 1302, resulting in moving the outer shaft 504 proximally (to expose valve and valve retainer 1004) or distally (to cover the valve 12 and valve retainer 1004). A retainer rod 1305, in turn, can be connected to the tether shaft 1208 (and can include a lumen through which the tether 78 can extend). An exit port 1307 in the handle 1300 for passage of the proximal end of the tether 78 is positioned just proximal to the retainer rod 1305. A nosecone shaft 1309 with a lumen therethrough (e.g., for a guidewire) can connect to hubs 1311, 1313, which can be fixed to the handle 1300. The handle 1300 can further include a two (or 1, 3, 4 or more) flush ports 1308. Additionally, the handle 1300 can include a plurality of seals (e.g., 2, 3, 4, 5, 6 or more).

In some embodiments, to deploy the valve frame 12 (e.g., from the valve delivery catheter 34 of FIG. 12) using the handle 1300, the deployment knob 1302 can be rotated to retract the outer shaft 504 proximally. As the outer shaft 504 is retracted proximally, the valve frame 12 can self-expand. In some embodiments, the valve prosthesis 12 can be retracted into the outer shaft 504 and repositioned and redeployed while the tabs 1008 are seated within the cutouts 1010 of the valve retainer 1004. The valve 12 can fully deployed when the valve retainer 1004 is fully exposed (via retraction of the outer shaft 504). In some embodiments, an additional retaining element, such as a suture, may be used to connect the valve prosthesis to the retainer 1004. The additional retaining element can be used to further control the release and expansion of the valve prosthesis.

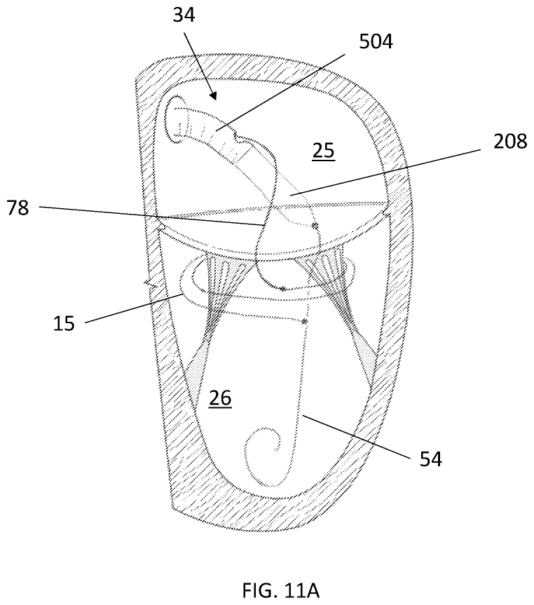

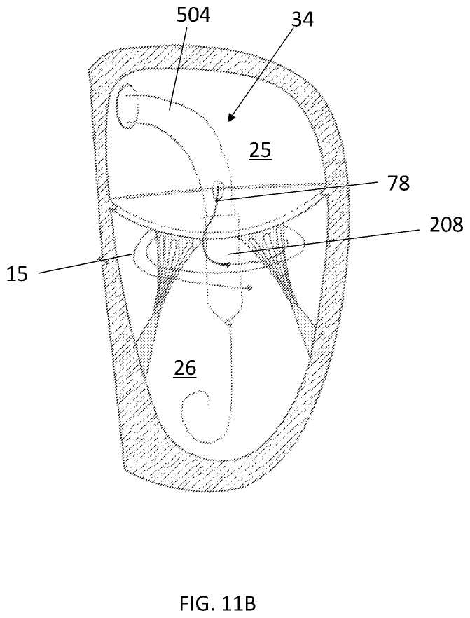

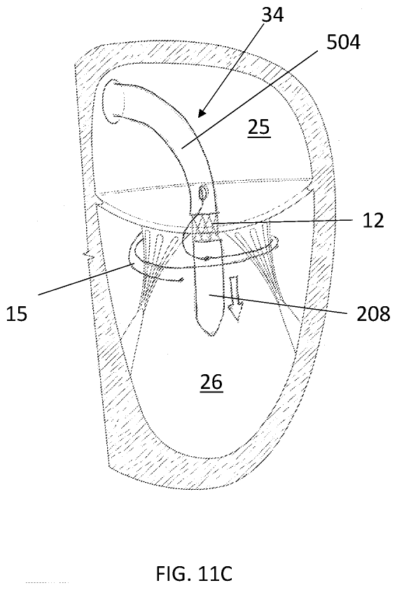

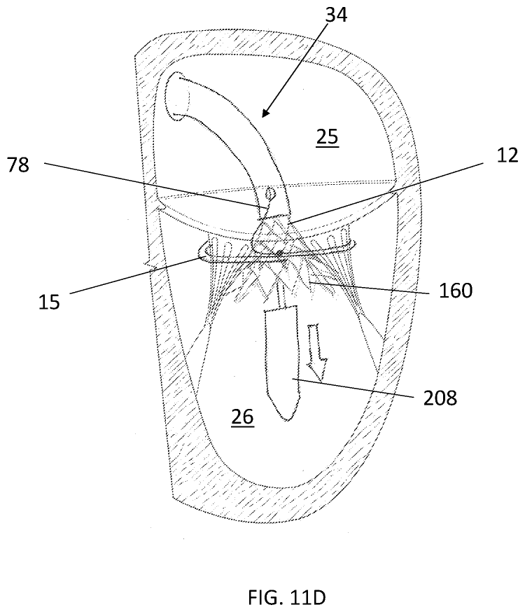

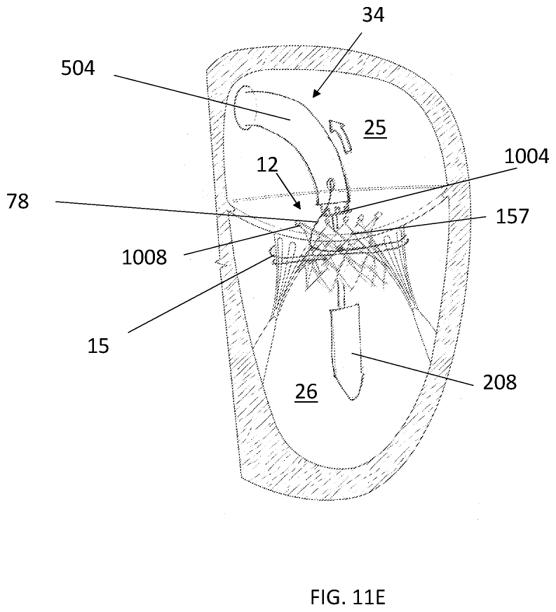

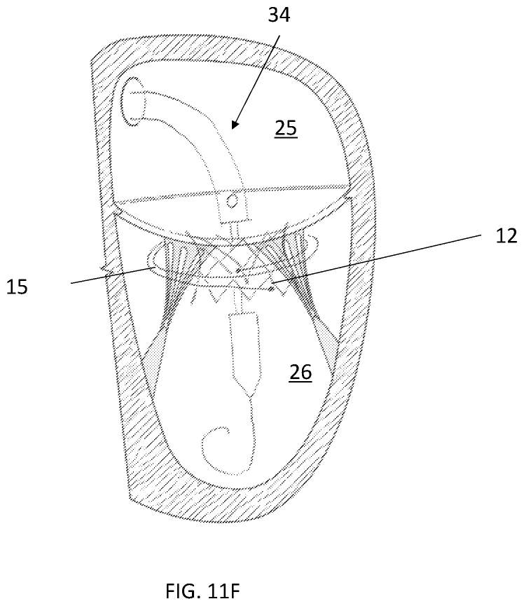

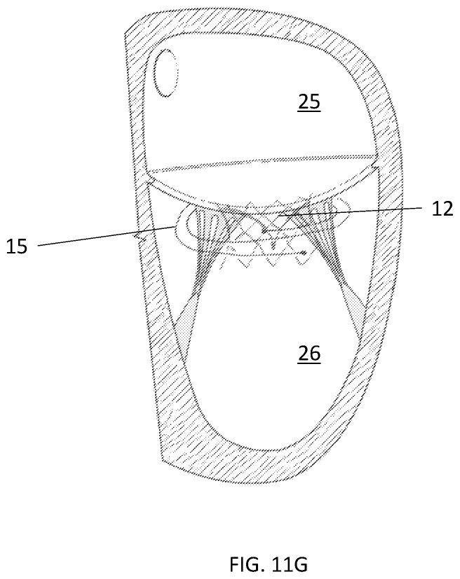

Referring to FIGS. 11A-11G, in some embodiments, the valve frame 12 can be held and released with a valve retainer 1004 and a distal valve capsule 208. In this embodiment, the distal (ventricular) portion of the valve frame 12 can be released by distal movement of the valve capsule 208 and the proximal (atrial) portion of the valve frame 12 can be released by proximal movement of the outer shaft 504 to expose the retainer 1004. FIGS. 11A-G show an exemplary method for delivering a valve frame 12 to a previously placed anchor 15. As shown in FIG. 11A, a valve delivery catheter 34 (including an outer sheath 504 and distal valve capsule 208) can tracked over a guidewire 54 into the atrium 25. A previously placed tether 78 is loaded into the valve delivery catheter 34 (e.g., as the valve delivery catheter 34 is tracked over the guidewire) to enable positional engagement between a previously placed anchor 15 and the valve delivery catheter 34. Referring to FIG. 11B, the valve delivery catheter 34 can pushed across the mitral valve annulus into the ventricle 26. Tension of the tether 78 can be managed (e.g., with a positioning tool as described herein) to achieve axial alignment of the anchor 15 and the valve frame 12. The anchor 15 can be pulled up towards the mitral annulus (e.g., with a positioning tool) to better engage with mitral leaflets and reduce paravalvular leakage. As shown in FIG. 11C, the valve capsule 208 can be moved distally relative to the outer shaft 504, beginning to expose valve frame 12. Referring to FIG. 11D, the valve capsule 208 can release a ventricular portion 160 of the valve frame 12. A deployment mechanism (e.g., positioning tool control or other proximal control mechanism) can maintain the axial height relationship between the valve, anchor, and anatomy as the valve capsule 208 translates distally. As shown in FIG. 11E, the outer sheath 504 can be translated proximally to expose the valve retainer 1004. The retainer 1004 can be configured to maintain the tabs 1008 of the valve frame until fully unsheathed by the outer shaft 504. Again, deployment mechanism (e.g., positioning tool control or other proximal control mechanism) can maintain the axial height relationship between the valve, anchor, and anatomy as the outer shaft 504 translates distally. Referring to FIG. 11F, the tether 78 can be disengaged from the anchor 15 and retracted into the valve delivery catheter 34. As shown in FIG. 11G, the valve delivery catheter 34 can be removed, leaving the anchor 15 and valve frame 12 in place.

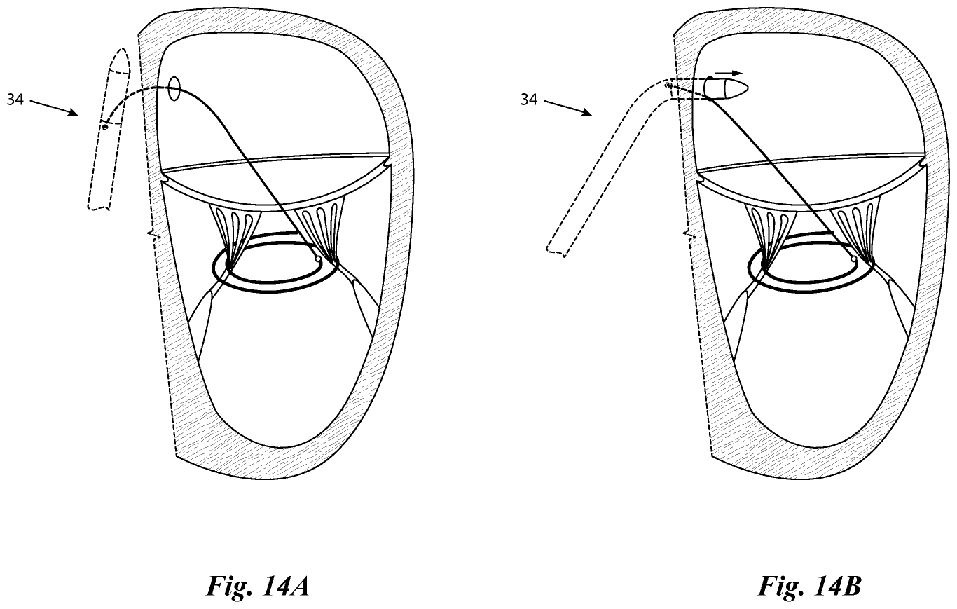

In some embodiments, the valve delivery catheter 34 tracks over the tether 78 and through the same septal puncture used to deliver the anchor. Further, as shown and described above, in some embodiments, the anchor tether port (e.g., the distal opening to the monorail channel 604 or the side port 204) is not located at the most distal extent of the valve delivery catheter 34. Rather, the distal opening to channel 604 or the side port 204 can instead be located on the side of the outer shaft 504, for example 1-2 inches proximal of the distal tip of the valve delivery catheter 34, as shown in and described with respect to FIGS. 4A-6K. This proximal setback can enable the functioning of the positioning tool 206 (e.g., by allowing the anchor positioning tool 206 to adjust the position of the anchor 15 such that it can be anywhere between the distal end of the frame 12 and the proximal end of the frame 12 when the frame 12 is in the valve delivery catheter 34). In some embodiments, however, the compliant nature of the septum, combined with the setback location of the distal opening to channel 604 or the side port 204, may cause the valve delivery catheter 34 to be deflected by the septum, which can cause septal crossing to be extremely challenging. FIGS. 14A and 14B show a failed septal crossing during which the valve delivery catheter 34 has been deflected by the septum (FIG. 14A), and a successful septal crossing (FIG. 14B).

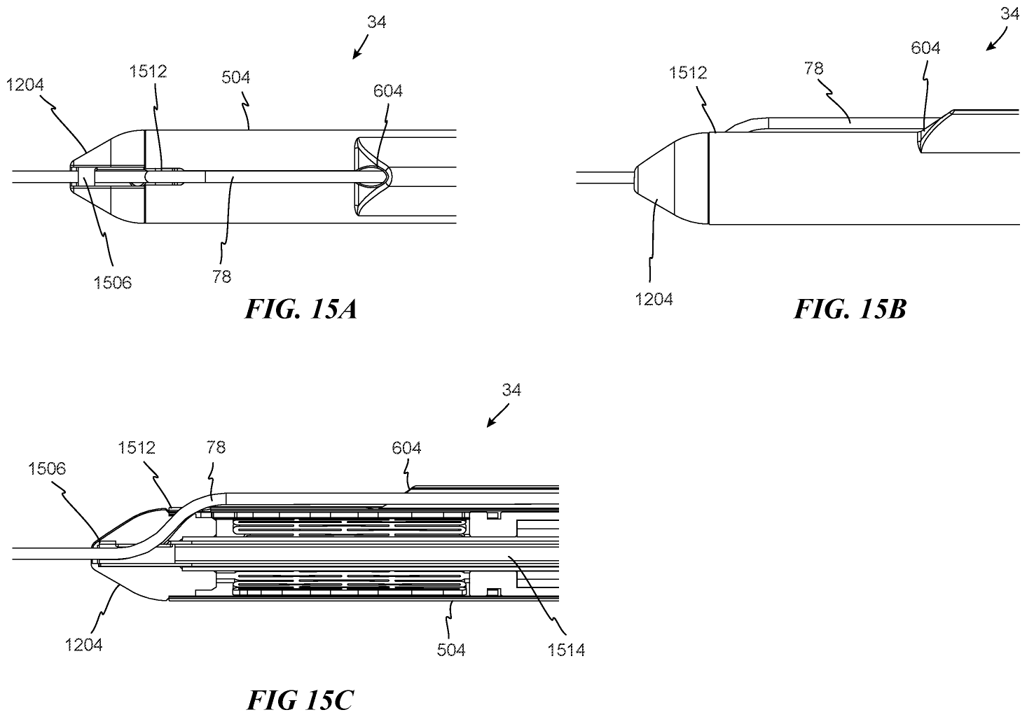

Referring to FIGS. 15A-C, to address these challenges, in some embodiments, the nosecone 1204 of the valve delivery catheter 34 can have a catch mechanism 1506. The tether 78 can thus extend from the monorail channel 604, along the outside of the outer shaft 504, and into a reentry port 1512 positioned between the nosecone 1204 and the monorail channel 604. A distal portion of the tether 78 can be positioned within a tether catch 1506 (shown closed) that is at the tip of the nosecone 1204 and on-axis with the outer shaft 504. This design can advantageously allow the tether 78 to be held at the center of the nosecone 1204 and valve delivery catheter 34 while advancing the valve delivery catheter 34 through the anatomy and across the septum. Once the septal crossing is complete, the tether 78 can be released from the nosecone 1204 by rotating the tether catch 1506 (with tether catch control shaft 1514) to an open position (i.e., such that an opening in the catch 1506 aligns with the reentry port 1512). Releasing the tether 78 from the nosecone 1204 allows the positioning tool 206 to adjust the position of the anchor 15 and also enables the release of the valve 12 from the valve delivery catheter 34.

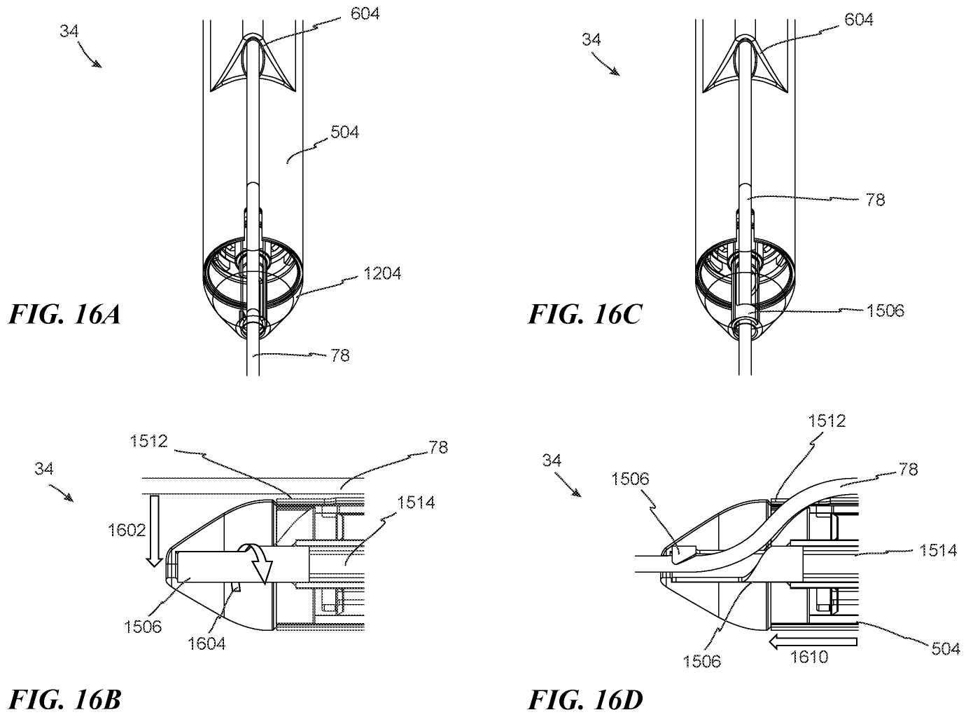

FIGS. 16A-16D show an exemplary method for catching and releasing the tether 78 using the catch mechanism shown in FIGS. 15A-15C. Before inserting the valve delivery catheter 34 into the body, the tether 78 can be inserted into the monorail channel 604. Once fully inserted, the tether 78 can be manually moved to the center of the nosecone 1204, as shown in the top perspective and side views of FIGS. 16A and 16B, respectively. Arrow 1602 of FIG. 16B indicates the direction in which the tether 78 is manually moved. The tether catch control shaft 1514 can be rotated counterclockwise to secure the tether 78 as shown by arrow 1604 in FIG. 16B. The top perspective and side views of FIGS. 16C and 16D, respectively, show the tether catch control shaft 1514 after it has secured the tether 78. Once the tether 78 is secured, the valve delivery catheter 34 can be advanced over the tether 78 by sliding the catch 1506 and the monorail channel 604 along the tether 78 (in the direction indicated by arrow 1610). The valve delivery catheter 34 can thus be passed over the tether 78 through the vasculature and across the septum. Once through the septum, the tether catch control shaft 1514 can be rotated clockwise (i.e., back to the position shown in FIG. 16A), releasing the tether 78.

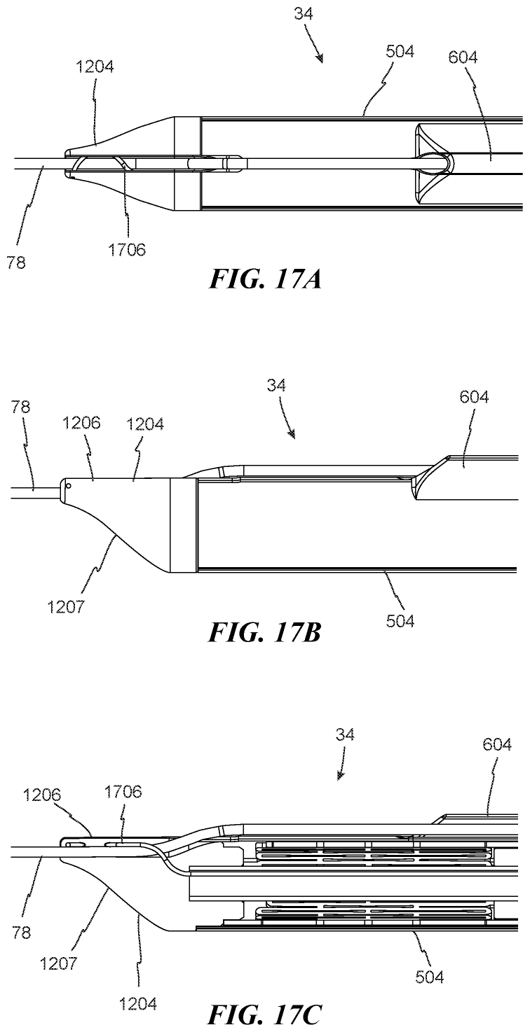

Referring now to FIGS. 17A-17C, another embodiment of a nosecone 1204 for catching and/or releasing the tether 78 is shown. The nosecone 1204 includes an axially offset distal tip. That is, the nosecone 1204 can include a first side 1206 that is substantially axially aligned with the wall of the outer shaft 504 (and/or aligned with distal opening to the monorail channel 604) and a second side 1207 that is angled relative to the outer shaft 504. Further, a retention wire 1706 can be used to hold the tether 78 within the nosecone 1204. The tether retention wire 1706 can be loaded and secured in place during manufacturing. In use, the tether retention wire 1706 can come free from the nosecone 1204 (releasing the tether 78) if sufficient axial tension is applied to the wire 1706 from the proximal end (e.g., at the handle). The axially offset nosecone 1204 of FIGS. 17A-17C can advantageously minimize the amount of bending that the tether 78 is subjected to.

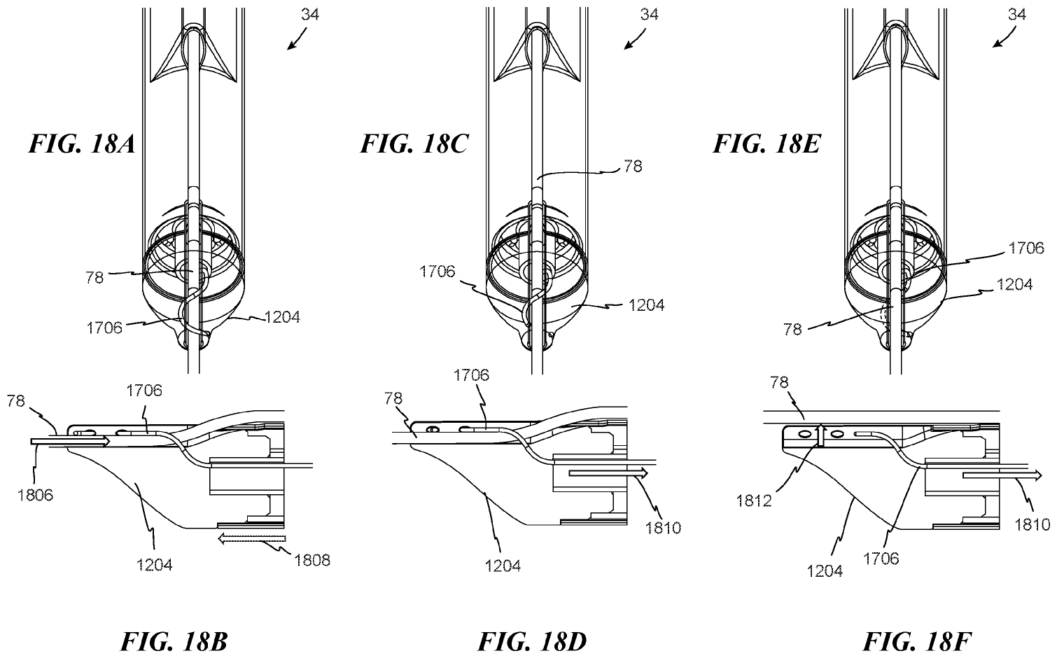

FIGS. 18A-18F show an exemplary method for catching and releasing the tether using the catch mechanism shown in FIGS. 17A-17C. As shown in FIGS. 18A and 18B, before inserting the valve delivery catheter 34 into the body, the tether 78 can be inserted through the nosecone 1204, passing under the retention wire 1706. Arrow 1806 (FIG. 18B) indicates the direction of insertion of the tether 78. The valve delivery catheter 34 can then be advanced over the tether 78, through the vasculature, and across the septum, as indicated by arrow 1808 (FIG. 18B). Referring to FIGS. 18C and 18D, once through the septum, the proximal end of the retention wire 1706 can be pulled proximally as indicated by arrow 1810. As shown in FIGS. 18E and 18F, once the retention wire 1706 has been pulled back sufficiently, the tether 78 can be released as indicated by arrow 1812.

The valve prosthesis described herein can include a frame structure and a valve segment. When described herein as a “frame structure,” it should be understood that the frame structure can include a valve segment therein. The valve segment may include leaflets formed of multi-layered materials for preferential function. The valve segment may comprise at least one leaflet having an inner layer and an outer layer. The valve segment may be attached directly to the frame structure. Alternatively, the valve segment may be attached to an intermediate valve structure that is in turn connected to the valve prosthesis. The valve segment may be connected to the frame structure before or after the valve prosthesis has been deployed adjacent a native valve. The frame structure may be attached to a leaflet of the valve segment, for example an outer layer of a leaflet, at one or more ends of the valve prosthesis. The frame structure may be attached to a leaflet of the valve segment, for example an outer layer of a leaflet, at one or more intermediate portions of the valve prosthesis. The valve segment may comprise a plurality of leaflets. The valve segment may comprise a biocompatible one-way valve. Flow in one direction may cause the leaflet(s) to deflect open and flow in the opposite direction may cause the leaflet(s) to close.

The frame structure may be configured like a stent. The frame structure may, for example, comprise a scaffold in a diamond pattern formed from a shape memory material (e.g., NiTi). One of ordinary skill in the art will appreciate that many other structures, materials, and configurations may be employed for the frame structure. For example, the frame structure may be formed of a polymer of sufficient elasticity. The frame structure may be formed of a combination of metal and polymer, such as metal (e.g., shape memory material) covered in polymer. The frame structure may include a variety of patterns besides diamond shapes. In some embodiments, frame structure is a closed frame such that blood flow is forced through valve segment therein. One or more skirts and/or seals may help force blood through valve segment.

It should be understood that any element(s) described herein with respect to one embodiment maybe combined with or substituted for any element(s) described with respect to another embodiment.

Although shown and described with respect to a mitral valve, one of ordinary skill in the art will understand that the principles described herein may be applied equally to other atrioventricular valves. Aspects of the methods, delivery systems, and valve prostheses described herein may include any of the elements described in International Application No. PCT/US2019/047542, filed on Aug. 21, 2019, titled “PROSTHETIC CARDIAC VALVE DEVICES, SYSTEMS, AND METHODS,” now International Publication No. WO 2020/041495, International Application No. PCT/US2019/055049, filed on Oct. 7, 2019, titled “PROSTHETIC CARDIAC VALVE DEVICES, SYSTEMS, AND METHODS,” now International Publication No. WO 2020/073050, International Application No. PCT/US2019/057082, filed on Oct. 18, 2019, titled “ADJUSTABLE MEDICAL DEVICE,” now International Publication No. WO 2020/082039, International Application No. PCT/US2019/068088, filed on Dec. 20, 2019, titled “PROSTHETIC CARDIAC VALVE DEVICES, SYSTEMS, AND METHODS,” now International Publication No. WO 2020/132590, International Application No. PCT/US2020/023671, filed on Mar. 19, 2020, titled “PROSTHETIC CARDIAC VALVE DEVICES, SYSTEMS, AND METHODS,” now International Publication No. WO 2020/191216, International Application No. PCT/US2020/027744, filed on Apr. 10, 2020, titled “MINIMAL FRAME PROSTHETIC CARDIAC VALVE DELIVERY DEVICES, SYSTEMS, AND METHODS,” now International Publication No. WO 2020/210685, International Application No. PCT/US2020/058413, filed on Oct. 30, 2020, titled “PROSTHETIC CARDIAC VALVE DEVICES, SYSTEMS, AND METHODS,” International Application No. PCT/US2021/021647, filed on Mar. 10, 2021, titled “PROSTHETIC CARDIAC VALVE DEVICES, SYSTEMS, AND METHODS,” and International Application No. PCT/US2021/020704, filed on Mar. 3, 2021, titled “PROSTHETIC CARDIAC VALVE DEVICES, SYSTEMS, AND METHODS,” the entireties of which are incorporated by reference herein.

One of ordinary skill in the art will recognize based on the description herein that any of the valve prostheses described herein may comprise any of the frame structure shapes, frame structure designs, frame structure materials, anchor shapes, anchor windings, anchor materials, free end tips, leaflet(s) configurations, or any other of the variable features described herein in any combination thereof as desired.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for treating a diseased native valve in a patient, the method comprising:
tracking a positioning tool in a first configuration and a valve delivery device over a tether into a heart, the tether connected to a spiral anchor in the heart;
activating the positioning tool from the first configuration to a second configuration that is stiffer than the first configuration;
adjusting, with the positioning tool, a position of the spiral anchor relative to a valve positioned within the delivery device; and
deploying a valve from the delivery device to an expanded position within the spiral anchor.

2. The method of claim 1, wherein the step of adjusting, with the positioning tool, a position of the spiral anchor relative to a valve positioned within the delivery device comprises pulling the positioning tool proximally.

3. The method of claim 1, further comprising advancing the positioning tool until a distal end of the positioning tool engages with a distal engagement element on the tether.

4. The method of claim 3, further comprising rotating the positioning tool until the distal end is clocked with respect to the distal engagement element.

5. The method of claim 1, wherein the step of activating the positioning tool from the first configuration to the second configuration comprises bending the positioning tool.

6. The method of claim 5, wherein bending the distal end of the positioning tool comprises tensioning the tether so as to compress the distal end of the positioning tool.

7. The method of claim 5, further comprising fixing a position of the positioning tool relative to a handle of the valve delivery device prior to bending the distal end of the positioning tool.

8. The method of claim 7, further comprising pulling proximally on the handle with the positioning tool fixed thereto to adjust a position of both the anchor and the valve relative to the heart.

9. The method of claim 1, wherein the step of adjusting, with the positioning tool, the position of the spiral anchor comprises moving the spiral anchor axially towards a native mitral valve annulus.

10. The method of claim 1, wherein the step of tracking a valve delivery device over a tether into a heart comprises tracking the valve delivery device over a tether into a first chamber of the heart.

11. The method of claim 10, further comprising advancing a distal end of the valve delivery device through a native valve annulus to a second chamber of the heart prior to deploying the valve.

12. The method of claim 1, wherein the step of tracking a valve delivery device over a tether into a heart comprises tracking a monorail lumen of the valve delivery device over the tether.

13. A delivery system for delivering and positioning a valve prosthesis within a diseased native valve, the delivery system comprising:
a valve delivery catheter;
a tether channel extending within or alongside the valve delivery catheter, wherein the tether channel is configured to receive a tether that is attached to a spiral anchor; and
a positioning tool configured to extend through the tether channel and over the tether, wherein the positioning tool comprises a first configuration and a second configuration, wherein a stiffness of the positioning tool in the second configuration is greater than a stiffness of the positioning tool in the first configuration.

14. The delivery system of claim 13, wherein the positioning tool in the second configuration comprises a fixed bend.

15. The delivery system of claim 13, wherein the positioning tool comprises a plurality of cutouts in a distal portion thereof, wherein the cutouts enable flexing of the positioning tool in the first configuration, and wherein the cutouts are fixed relative to one another in the second configuration.

16. The delivery system of claim 13, wherein a distal engagement element of the tether is configured to compress the positioning tool to place or maintain the positioning tool in the second configuration.

17. The delivery system of claim 16, wherein the distal engagement element is beveled.

18. The delivery system of claim 16, further comprising a proximal control comprising an actuator configured to control tension on the tether so as to compress the positioning tool.

19. The delivery system of claim 18, wherein the actuator comprises a knob configured to enable sliding of a tether clamp relative to the positioning tool.

20. The delivery system of claim 13, further comprising a proximal control comprising a rotation mechanism configured to rotationally orient the positioning tool with respect to the tether.

21-100. (canceled)
